(19) United States
(12) Patent Application Publication
(10) Pub. No.: US 2026/0028647 A1
(43) Pub. Date: Jan. 29, 2026

(54) Compositions and Methods for Editing Cytoplasmic DNA

(71) Applicant: The Regents of the University of California, Oakland, CA (US)

(72) Inventors: Adam Joseph Schieferecke, Oakland, CA (US); David V. Schaffer, Oakland, CA (US); John E. Dueber, Oakland, CA (US); Juan Eduardo Hurtado Koolik, Oakland, CA (US)

(21) Appl. No.: 18/875,658

(22) PCT Filed: Jul. 6, 2023
(86) PCT No.: PCT/US2023/069704

§ 371 Date: Dec. 16, 2024

Related U.S. Application Data
(60) Provisional application No. 63/358,963, filed on Jul. 7, 2022.

(51) Int. Cl. C12N 15/90; C12N 9/22; C12N 15/11

(52) U.S. Cl. CPC C12N 15/902; C12N 9/226; C12N 15/11; C07K 2319/095; C12N 2310/20

(57) ABSTRACT

The present disclosure provides systems and methods for modifying a target viral nucleic acid in the cytoplasm of a eukaryotic cell.

### CROSS-REFERENCE

This application claims the benefit of PCT/US2023/069704, filed Jul. 6, 2023, which application claims the benefit of U.S. Provisional Patent Application No. 63/358,963, filed Jul. 7, 2022, which applications are incorporated herein by reference in their entirety.

### INCORPORATION-BY-REFERENCE OF MATERIAL ELECTRONICALLY SUBMITTED

A Sequence Listing is provided herewith as a Sequence Listing XML, “BERK-470_SEQ_LIST_SEP_2025” created on Sep. 15, 2025 and having a size of 119,414 bytes. The contents of the Sequence Listing XML are incorporated by reference herein in their entirety.

### INTRODUCTION

Nucleocytoplasmic large DNA viruses (NCLDVs) are a group of viruses that harbor large (150 kbp-1.2 mbp) double-stranded DNA genomes and replicate in the cytoplasm of eukaryotic cells. NCLDVs encompass multiple viral families, including Poxviridae, Asfaviridae, Iridoviridae, Ascovirida, Phycodnaviridae, Marseilleviridae, Pithoviridae, and Mimiviridae, as well as Pandoraviruses, Molliviruses, and Faustoviruses. Millions of years of natural evolution resulted in the capability of NCLDVs to deliver exogenous nucleic acids and proteins to cells. Certain viral species belonging to NCLDV families have been adapted for use as recombinant vaccines, immunotherapies, adjuvants, gene therapies, expression systems for protein manufacturing, pest control agents, and in other biotechnologically useful applications. However, natural evolution resulted in qualities that are not optimal for many applications. Directed evolution, the iterative process of creating diversity followed by screening for genotypes that confer improved function, is a powerful approach that has been used to engineer improved genetically encoded molecules, including increasingly complex systems such as small virus-based gene delivery systems. However, large genome sizes, repetitive sequence regions, cytoplasmic replication, limited homologous recombination efficiencies, and reliance upon unique viral promoters and replication machinery present barriers to applying directed evolution approaches to engineering NCLDVs using existing techniques.

Clustered Regularly Interspaced Short Palindromic Repeats (CRISPR)-Cas systems comprise a CRISPR-associated (Cas) effector polypeptide and a guide nucleic acid. Such CRISPR-Cas systems can bind to and modify a targeted nucleic acid. The programmable nature of these CRISPR-Cas effector systems has facilitated their use as a versatile technology for use in, e.g., gene editing.

There is a need in the art for compositions and methods for mutagenizing large DNA viruses in the cytoplasm of a eukaryotic cell.

### SUMMARY

The present disclosure provides systems and methods for modifying a target viral nucleic acid in the cytoplasm of a eukaryotic cell.

### BRIEF DESCRIPTION OF THE DRAWINGS

FIG. 1A-1F provide amino acid sequences of DNA Polymerase I (PolI) and variants (SEQ ID NOs: 1-6, respectively).

FIG. 2 provides an amino acid sequence of Phi29 DNA polymerase (SEQ ID NO:7).

FIG. 3 provides an amino acid sequence of a T5 DNA polymerase (SEQ ID NO:8).

FIG. 4A-4B provide amino acid sequences of T7 DNA polymerase and Sequenase (SEQ ID NOs: 9-10, respectively).

FIG. 5 provides an amino acid sequence of DNA polymerase Iota (SEQ ID NO: 11).

FIGS. 6A-6B provide an amino acid sequence of DNA polymerase η (SEQ ID NO:12).

FIGS. 7A-7B provide an amino acid sequence of DNA polymerase κ (SEQ ID NO:13).

FIGS. 8A-8D provide an amino acid sequence of DNA polymerase θ (SEQ ID NO:14).

FIGS. 9A-9B provide an amino acid sequence of DNA polymerase v (SEQ ID NO: 15).

FIG. 10A-10L provide amino acid sequences of CRISPR-Cas effector polypeptides (SEQ ID NOs: 16-27, respectively).

FIG. 11A-11E depict a system for characterizing diversification of user-defined loci in cytoplasmic DNA using the poxvirus vaccinia as a model (BFP amino acid and nucleic acid sequence: SEQ ID NOs: 28-29, respectively) (GFP amino acid and nucleic acid sequence: SEQ ID NOs: 30-31, respectively).

FIG. 12A-12C depict data showing that truncation of PAM-distal base pairs from a single-guide RNA (sgRNA) template-binding region increases nSpRY-Pol15M-mediated effective single nucleotide polymorphism (SNP) generation.

FIG. 13A-13B depict data showing that nSpRY-Pol15M guided by full-length (20 bp target site-binding) sgRNAs and truncated (18 bp target site-binding) sgRNAs, respectively; can target an AT-rich region of the A34R gene of vaccinia virus to create site-specific diversity.

FIG. 14A-14D provides amino acid sequences of Cas9 variants (SEQ ID NOs: 88-90 and 32, respectively).

FIG. 15 depicts data showing an RNA-guided nSpRY-PolI5M fusion complex conferred on-target mutagenesis of VV-BFP with low off-target effects.

FIG. 16 depicts data showing an RNA-guided nSpRY-PolI5M fusion complex conferred on-target mutagenesis in the genome of a distantly related poxvirus species, myxoma virus.

FIG. 17 depicts data showing a miniaturized nuclease and polymerase fusion protein complexed with truncated (18 bp target site-binding) gRNAs generated elevated diversity at a targeted locus in VV-BFP.

FIG. 18 depicts data showing the nSpRY-PolI5M fusion protein guided by a pool of 39 sgRNAs generates elevated diversity across an endogenous gene of interest.

FIG. 19 provides an amino acid sequence of a truncated nSpRY-Cas9 variant (SEQ ID NO: 32).

FIG. 20 provides a nucleic acid sequence of a truncated PolI5M variant (SEQ ID NO: 33).

### Definitions

“Heterologous,” as used herein in the context of a polypeptide, refers to an amino acid sequence that is not found in the native polypeptide. For example, a fusion CRISPR-Cas effector polypeptide comprises: a) a CRISPR-Cas effector polypeptide; and b) one or more heterologous polypeptides, where the heterologous polypeptide comprises an amino acid sequence from a protein other than a CRISPR-Cas effector polypeptide. “Heterologous.” as used herein in the context of a nucleic acid, refers to a nucleotide sequence that is not found in the native nucleic acid. As an example, in a guide nucleic acid, a heterologous guide nucleotide sequence (present in a targeting segment) that can hybridize with a target nucleotide sequence (target region) of a target nucleic acid is a nucleotide sequence that is not found in nature in a guide nucleic acid together with a binding segment that can bind to a CRISPR-Cas effector polypeptide. For example, in some cases, a heterologous target nucleotide sequence (present in a heterologous targeting segment) is from a different source than a binding nucleotide sequence (present in a binding segment) that can bind to a CRISPR-Cas effector polypeptide of the present disclosure. For example, a guide nucleic acid may comprise a guide nucleotide sequence (present in a targeting segment) that can hybridize with a target nucleotide sequence present in a eukaryotic target nucleic acid. A guide nucleic acid of the present disclosure can be generated by human intervention and can comprise a nucleotide sequence not found in a naturally-occurring guide nucleic acid.

The term “naturally-occurring” as used herein as applied to a nucleic acid, a protein, a cell, or an organism, refers to a nucleic acid, cell, protein, or organism that is found in nature.

The terms “polynucleotide” and “nucleic acid.” used interchangeably herein, refer to a polymeric form of nucleotides of any length, either ribonucleotides or deoxynucleotides or combinations thereof. Thus, this term includes, but is not limited to, single-, double-, or multi-stranded DNA or RNA, genomic DNA, cDNA. DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

As used herein, the term “guide RNA” (gRNA) and the like refer to an RNA that guides a CRISPR-Cas effector polypeptide (or a fusion protein comprising a CRISPR-Cas effector polypeptide) to a target sequence in a target nucleic acid. The term gRNA can also refer to a prime editing guide RNA (pegRNA), a nicking guide RNA (ngRNA), and a single guide RNA (sgRNA). In some cases, the term “gRNA molecule” refers to a nucleic acid encoding a gRNA. In some cases, the gRNA molecule is naturally occurring. In some cases, a gRNA molecule is non-naturally occurring. In some cases, a gRNA molecule is a synthetic gRNA molecule.

The terms “polypeptide.” “peptide.” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence.

Polypeptides as described herein also include polypeptides having various amino acid additions, deletions, or substitutions relative to the native amino acid sequence of a polypeptide of the present disclosure. In some embodiments, polypeptides that are homologs of a polypeptide of the present disclosure contain non-conservative changes of certain amino acids relative to the native sequence of a polypeptide of the present disclosure. In some embodiments, polypeptides that are homologs of a polypeptide of the present disclosure contain conservative changes of certain amino acids relative to the native sequence of a polypeptide of the present disclosure, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (sec, e.g., Creighton. Proteins (1984)). A modification of an amino acid to produce a chemically similar amino acid may be referred to as an analogous amino acid.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990). J. Mol. Biol. 215:403-10. Another alignment algorithm is FASTA, available in the Genetics Computing Group (GCG) package, from Madison. Wisconsin. USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle. Academic Press. Inc., a division of Harcourt Brace & Co., San Diego, California. USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See J. Mol. Biol. 48:443-453 (1970).

The terms “DNA regulatory sequences.” “control elements.” and “regulatory elements.” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (e.g., DNA exogenous to the cell) into the cell. Genetic change (“modification”) can be accomplished either by incorporation of the new nucleic acid into the genome of the host cell, or by transient or stable maintenance of the new nucleic acid as an episomal element. Where the cell is a eukaryotic cell, a permanent genetic change is generally achieved by introduction of new DNA into the genome of the cell.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a.” “an.” and “the” include plural referents unless the context clearly dictates otherwise. Thus, for example, reference to “a single-guide RNA” includes a plurality of such single-guide RNAs and reference to “the error-prone DNA polymerase” includes reference to one or more error-prone DNA polymerases and equivalents thereof known to those skilled in the art, and so forth. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation.

The use of the terms “a.” “an.” and “the,” and similar referents in the context of describing the disclosure (especially in the context of the following claims) are to be construed to cover both the singular and the plural, unless otherwise indicated herein or clearly contradicted by context. The terms “comprising.” “having.” “including.” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any nonclaimed element as essential to the practice of the embodiments of the disclosure.

As used herein, the term “about” used in connection with an amount indicates that the amount can vary by 10% of the stated amount. For example. “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The term “and/or” as used herein a phrase such as “A and/or B” is intended to include both A and B: A or B; A (alone); and B (alone). Likewise, the term “and/or” as used herein a phrase such as “A, B, and/or C” is intended to encompass each of the following embodiments; A, B, and C; A, B, or C; A or C; A or B; B or C; A and C; A and B; B and C; A (alone); B (alone); and C (alone).

It is understood that aspects and embodiments of the present disclosure described herein include “comprising.” “consisting.” and “consisting essentially of” aspects and embodiments.

It is appreciated that certain features of the invention, which are, for clarity, described in the context of separate embodiments, may also be provided in combination in a single embodiment. Conversely: various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides systems and methods for modifying a target viral nucleic acid in the cytoplasm of a eukaryotic cell.

The present disclosure provides a method of mutagenizing user-defined regions of cytoplasmic DNA using a single guide RNA (sgRNA) or combinations of sgRNAs and a highly engineered fusion polypeptide, where the fusion polypeptide comprises: a) an enzymatically active, RNA-guided endonuclease that introduces a single-stranded break in cytoplasmic DNA; and b) an error-prone DNA polymerase. In some cases, the fusion polypeptide comprises: a) an enzymatically active, RNA-guided endonuclease that introduces a single-stranded break in cytoplasmic DNA; b)) a nuclear export sequence (NES); and c) an error-prone DNA polymerase. In some cases, the fusion polypeptide does not include a nuclear localization signal (NLS) polypeptide.

### Methods for Modifying a Target Nucleic Acid in the Cytoplasm of a Eukaryotic Cell

The present disclosure provides methods for modifying a target viral nucleic acid in the cytoplasm of a eukaryotic cell. In some cases, the methods comprise contacting the target viral nucleic acid with: a) a fusion polypeptide comprising: i) a CRISPR-Cas effector polypeptide, where the CRISPR-Cas effector polypeptide is a nickase (i.e., the CRISPR-Cas effector polypeptide introduces a single-stranded break in the target viral nucleic acid); and ii) two or more heterologous polypeptides, wherein one of the two or more heterologous polypeptides is an error-prone DNA polymerase and wherein one of the two or more heterologous polypeptides comprises a nuclear export signal (NES) polypeptide; and b) one or more guide nucleic acids, wherein one or more guide nucleic acids comprise: i) a targeting region that comprises a nucleotide sequence that binds to a target sequence in the target viral nucleic acid; and ii) a protein-binding region that binds to the CRISPR-Cas effector polypeptide. In some cases, the methods comprise contacting the target viral nucleic acid with: a) a fusion polypeptide comprising: i) a CRISPR-Cas effector polypeptide, where the CRISPR-Cas effector polypeptide is a nickase (i.e., the CRISPR-Cas effector polypeptide introduces a single-stranded break in the target viral nucleic acid); and ii) one or more heterologous polypeptides, wherein one of the one or more heterologous polypeptides is an error-prone DNA polymerase; and b) one or more guide nucleic acids, wherein one or more guide nucleic acids comprise: i) a targeting region that comprises a nucleotide sequence that binds to a target sequence in the target viral nucleic acid; and ii) a protein-binding region that binds to the CRISPR-Cas effector polypeptide. Contacting the target viral nucleic acid with the fusion polypeptide and the one or more guide nucleic acids provides for modification of the target viral nucleic acid. In some cases, the fusion polypeptide does not include a nuclear localization signal (NLS) polypeptide.

In some cases, the fusion polypeptide, when complexed with a guide RNA, exhibits a target mutation rate of from 10⁻⁸ to 10⁻² mutations per nucleotide per viral genome replication event. In some cases, the fusion polypeptide, when complexed with a guide RNA, exhibits a target mutation rate of greater than 10⁻⁸ mutations per nucleotide per viral genome replication event, e.g., greater than 10⁻⁸, greater than 10⁻⁷, greater than 10⁻⁶, greater than 10⁻⁵, greater than 10⁻⁴, or greater than 10⁻³, mutations per nucleotide per viral genome replication event. In some cases, the fusion polypeptide, when complexed with a guide RNA, exhibits a target mutation rate of from 10⁻⁸ to 10⁻⁷ mutations per nucleotide per viral genome replication event. In some cases, the fusion polypeptide, when complexed with a guide RNA, exhibits a target mutation rate of from 10⁻⁷ to 10⁻⁶ mutations per nucleotide per viral genome replication event. In some cases, the fusion polypeptide, when complexed with a guide RNA, exhibits a target mutation rate of from 10⁻⁷ to 10⁻⁵ mutations per nucleotide per viral genome replication event. In some cases, the fusion polypeptide, when complexed with a guide RNA, exhibits a target mutation rate of from 10⁻⁵ to 10⁻⁴ mutations per nucleotide per viral genome replication event. In some cases, the fusion polypeptide, when complexed with a guide RNA, exhibits a target mutation rate of from 10⁻⁴ to 10⁻³ mutations per nucleotide per viral genome replication event. In some cases, the fusion polypeptide, when complexed with a guide RNA, exhibits a target mutation rate of from 10⁻³ to 10⁻² mutations per nucleotide per viral genome replication event. In some cases, the fusion polypeptide, when complexed with a guide RNA, exhibits a target mutation rate of from 10⁻² to 10⁻¹ mutations per nucleotide per viral genome replication event.

In some cases, a fusion polypeptide, when complexed with a guide RNA, exhibits a target mutation rate of 1 mutation per nucleotide per viral genome replication event.

In some cases, a fusion polypeptide, when complexed with a guide RNA, exhibits a ratio of target mutation rate to global mutation rate of at least 1.5:1, at least 2:1, at least 5:1, at least 10:1, at least 25:1, at least 50:1, at least 10²:1, at least 5×10²:1, at least 10³:1, at least 5×10³:1, at least 10⁴:1, or more than 10⁴:1. In some cases, a fusion polypeptide, when complexed with a guide RNA, exhibits a ratio of target mutation rate to global mutation rate of from about 1.5:1 to 10⁴:1, e.g., from about 1.5:1 to 2:1, from 2:1 to 5:1, from 5:1 to 10:1, from 10:1 to 25:1, from 25:1 to 50:1, from 50:1 to 10²:1, from 10²:1 to 5×10²:1, from 5×10²:1 to 10³:1, from 10³:1 to 5×10³:1, from 5×10³:1 to 10⁴:1, or more than 10⁴:1.

In some cases, a fusion polypeptide, when complexed with a guide RNA, exhibits a target mutation rate that is at least 2-fold higher than the target mutation rate exhibited by the error-prone DNA polymerase present in the fusion polypeptide when the error-prone DNA polymerase is not fused to the CRISPR-Cas effector polypeptide present in the fusion polypeptide. In some cases, a fusion polypeptide, when complexed with a guide RNA, exhibits a target mutation rate that is at least 2-fold, at least 5-fold, at least 10-fold, at least 50-fold, at least 10²-fold, at least 5×10²-fold, at least 10³-fold, at least 5×10³-fold, or at least 10⁴-fold, higher than the target mutation rate exhibited by the error-prone DNA polymerase present in the fusion polypeptide when the error-prone DNA polymerase is not fused to the CRISPR-Cas effector polypeptide present in the fusion polypeptide. In some cases, a fusion polypeptide, when complexed with a guide RNA, exhibits a target mutation rate that is more than 10⁴-fold higher than the target mutation rate exhibited by the error-prone DNA polymerase present in the fusion polypeptide when the error-prone DNA polymerase is not fused to the CRISPR-Cas effector polypeptide present in the fusion polypeptide.

In some cases, a fusion polypeptide, when complexed with a guide RNA, introduces mutations at a distance of from I nucleotide to 10⁴ nucleotides from a nick in a target DNA introduced by the CRISPR-Cas effector polypeptide. For example, in some cases, a fusion polypeptide, when complexed with a guide RNA, introduces mutations at a distance of from 1 nucleotide (nt) to 10 nucleotides (nt), from 10 nt to 50 nt, from 50 nt to 100 nt, from 100 nt to 500 nt, from 500 nt to 10³ nt, from 10³ nt to 5×10³ nt, or from 5×10³ nt to 10⁴ nt from a nick in a target DNA introduced by the CRISPR-Cas effector polypeptide. In some cases, a fusion polypeptide, when complexed with a guide RNA, introduces mutations at a distance of from 1 nt to 10 nt from a nick in a target DNA introduced by the CRISPR-Cas effector polypeptide. In some cases, a fusion polypeptide, when complexed with a guide RNA, introduces mutations at a distance of from 1 nt to 25 nt from a nick in a target DNA introduced by the CRISPR-Cas effector polypeptide. In some cases, a fusion polypeptide, when complexed with a guide RNA, introduces mutations at a distance of from 10 nt to 25 nt from a nick in a target DNA introduced by the CRISPR-Cas effector polypeptide. In some cases, a fusion polypeptide, when complexed with a guide RNA, introduces mutations at a distance of from 1 nt to 50 nt from a nick in a target DNA introduced by the CRISPR-Cas effector polypeptide. In some cases, a fusion polypeptide, when complexed with a guide RNA, introduces mutations at a distance of from 10 nt to 50 nt from a nick in a target DNA introduced by the CRISPR-Cas effector polypeptide. In some cases, a fusion polypeptide, when complexed with a guide RNA, introduces mutations at a distance of from 25 nt to 50 nt from a nick in a target DNA introduced by the CRISPR-Cas effector polypeptide. In some cases, a fusion polypeptide, when complexed with a guide RNA, introduces mutations at a distance of from 1 nt to 100 nt from a nick in a target DNA introduced by the CRISPR-Cas effector polypeptide. In some cases, a fusion polypeptide, when complexed with a guide RNA, introduces mutations at a distance of from 10 nt to 100 nt from a nick in a target DNA introduced by the CRISPR-Cas effector polypeptide. In some cases, a fusion polypeptide, when complexed with a guide RNA, introduces mutations at a distance of from 50 nt to 100 nt from a nick in a target DNA introduced by CRISPR-Cas effector polypeptide.

In some cases, the fusion polypeptide has a length of no more than about 3000 amino acids. In some cases, the fusion polypeptide has a length of from about 1000 amino acids to about 3000 amino acids. In some cases, the fusion polypeptide has a length of from about 1000 amino acids to about 1250 amino acids, from about 1250 amino acids to about 1500 amino acids, from about 1500 amino acids to about 1750 amino acids, from about 1750 amino acids to about 2000 amino acids, from about 2000 amino acids to about 2250 amino acids, from about 2250 amino acids to about 2500 amino acids, from about 2500 amino acids to about 2750 amino acids, or from about 2750 amino acids to about 3000 amino acids.

Mutations that can be introduced into a target viral nucleic acid include insertions, deletions, substitutions, and the like.

### Target Viral Nucleic Acids

Viral nucleic acids that can be modified using a method of the present disclosure are referred to as “target viral nucleic acids.” A suitable target viral nucleic acid is a double-stranded DNA virus that has a genome length of from about 50 kilo base pairs (kbp) to about 1.2 mega base pairs (mbp), where at least part of the replication cycle of the double-stranded DNA virus occurs in the cytoplasm of the cell. Such viruses are sometimes referred to as “nucleocytoplasmic large DNA viruses” or “NCLDVs”. In some cases, a suitable target viral nucleic acid is a double-stranded DNA virus that has a genome length of from about 50 kbp to 150 kbp, from about 150 kbp to about 500 kbp, from about 500 kbp to about 1000 kbp, or from about 1000 kbp to about 1.2 mbp.

NCLDVs encompass multiple viral families, including Poxviridae, Asfaviridae, Iridoviridae, Ascoviridae, Phycodnaviridae, Marseilleviridae, Pithoviridae, Mimiviridae,

### Pandoraviridae, Mininucleoviridae, Molliviruses, and Faustoviruses.

In some cases, the target viral nucleic acid is a member of Poxviridae. Poxiviridae includes the genuses Avipoxvirus, Capripoxvirus, Centapoxvirus, Cervidpoxvirus, Crocodylidpoxvirus, Leporipoxvirus, Macroopoxvirus, Molluscipoxvirus, Mustelpoxvirus, Orthopoxivirus, Oryzopoxvirus, Parapoxvirus, Pteropopoxvirus, Scieuripoxvirus, Suipoxvirus, Vespertilionpoxvirus, Yatapoxvirus, Alphaentemopoxvirus, Betaentemopoxvirus, Deltaentomopoxvirus, Diachasmimorphaentemopoxvirus, and Gammaentomopoxvirus. The Orthopoxvirus genus includes vaccinia virus, cowpox virus, monkeypox virus, and rabbitpox virus. In some cases, the target viral nucleic acid is a nucleic acid of a Myxoma virus. Squirrel Fibroma Virus, or an Ectromelia virus. In some cases, the target viral nucleic acid is a vaccinia virus. In some cases, the target viral nucleic acid is a vaccinia virus of any one of the following vaccinia virus strains: 1) Western Reserve; 2) Wyeth; 3) New York City Board of Health (NYCBH); 4) Paris; 5) Acambis 2000; 6) Bern; 7) Ankara; 8) IHD-J; 9) Copenhagen (Cop); 10) Temple of Heaven; 11) Dairen; 12) Lister; 13) Tian Tan; 14) Modified Vaccinia Ankara (MVA); 15) Lister clone 16m8 (LC16m8); and 16) Dairen I (DIs). In some cases, the target viral nucleic acid is a nucleic acid of a chimeric poxvirus strain or a recombinant viral strain encoding heterologous DNA.

In some cases, a target nucleotide sequence in a target viral nucleic acid is a coding sequence, e.g., the target nucleotide sequence encodes a polypeptide and/or an RNA. A target nucleotide sequence in a target viral nucleic acid can be a nucleotide sequence in a coding sequence that encodes a polypeptide such as a polypeptide that is involved in dissemination of the virus, an antigenic polypeptide (e.g., a polypeptide in the viral capsid), a polypeptide that provides for oncolytic activity, a polypeptide that functions in release of a virus from a cell, a polypeptide that provides for infectivity of a virus, a polypeptide that alters the cell or host specificity of a virus, a polypeptide that alters the entry mechanism of a virus, a polypeptide that alters the intracellular trafficking of a virus, a polypeptide that alters the actin-based propulsion of a virus in intra- or extra-cellular environments, a polypeptide that alters microtubule association or trafficking of a virus, a polypeptide involved in the formation and maturation of intracellular mature virion (IMV), a polypeptide involved in the formation of cell-associated enveloped virion (CEV), a polypeptide involved in the formation of extracellular enveloped virus (EEV), a virally encoded polypeptide that functions in antagonizing the host innate or adaptive antiviral immune response, and the like.

Any vaccinia virus gene can be a target nucleic acid. For example, vaccinia virus coding regions that may be of interest as target nucleotides include a vaccinia virus gene selected from F13L. A36R, A34R, A53R, B5R, B7R, B13R, B15R, B22R, B28R, B29R, A33R, B8R, B18R, SPI-1, SPI-2, B15R, CUR, VGF, E3L, K2L, K3L, A41L, K7R, vC12L, vCKBP, and NIL. For example A34R is a vaccinia virus glycoprotein required for cellular release and infectivity of EEV, B5R, F13L, A36R, A34R, and A33R are examples of EEV-specific membrane proteins.

In some cases, a method of the present disclosure provides for introduction into a target viral nucleic acid one or more mutations, thereby generating a variant virus, where the variant virus exhibits increased oncolytic activity compared to the unmutated virus (i.e., a control virus that does not include the one or more mutations but is otherwise identical to the variant virus). In some cases, a method of the present disclosure provides for introduction into a target viral nucleic acid one or more mutations, thereby generating a variant virus, where the variant virus exhibits oncolytic activity that is at least 10%, at least 25%, at least 50%, at least 100% (or 2-fold), at least 5-fold, at least 10-fold, or more than 10-fold, higher than the oncolytic activity of the unmutated virus (i.e., a control virus that does not include the one or more mutations but is otherwise identical to the variant virus).

In some cases, a method of the present disclosure provides for introduction into a target viral nucleic acid one or more mutations, thereby generating a variant virus, where the variant virus exhibits increased production of EEV compared to the unmutated virus (i.e., a control virus that does not include the one or more mutations but is otherwise identical to the variant virus). In some cases, a method of the present disclosure provides for introduction into a target viral nucleic acid one or more mutations, thereby generating a variant virus, where the variant virus exhibits at least 10%, at least 25%, at least 50%, at least 100% (or 2-fold), at least 5-fold, at least 10-fold, or more than 10-fold, greater production of EEV, compared to the unmutated virus (i.e., a control virus that does not include the one or more mutations but is otherwise identical to the variant virus).

In some cases, a method of the present disclosure provides for introduction of one or more mutations into a target viral nucleic acid, thereby generating a variant virus, where the variant virus exhibits reduced neutralization by neutralizing antibodies in a mammalian host (e.g., a human). In some cases, a method of the present disclosure provides for introduction of one or more mutations into a target viral nucleic acid, thereby generating a variant virus, where the variant virus exhibits one or more of: 1) increased virion production in target cells: 2) increased CEV formation: 3) increased EEV formation: 4) modified mechanism of cell entry conferring altered cell tropism: 5) increased intracellular trafficking: 6) increased kinetics of viral replication, maturation, and egress; and 6) improved dissemination among metastasized tumor cells within a human or non-human animal.

In some cases, a method of the present disclosure comprises introducing into a eukaryotic cell in vitro: i) a recombinant expression vector comprising a nucleotide sequence encoding the fusion polypeptide (i.e., a fusion polypeptide comprising: i) a CRISPR-Cas effector polypeptide; and ii) two or more heterologous polypeptides, wherein one of the two or more heterologous polypeptides is an error-prone DNA polymerase and wherein one of the two or more heterologous polypeptides comprises an NES polypeptide); and ii) one or more guide RNAs. In some cases, the guide RNA is a single-molecule guide RNA (a “sgRNA”); i.e., where the guide RNA is a single RNA molecule. In some cases, the nucleotide sequence encoding the fusion protein is operably linked to a promoter. In some cases, the promoter is a constitutive promoter. In some cases, the promoter is a regulatable (e.g., inducible) promoter.

In some cases, a method of the present disclosure comprises introducing into a eukaryotic cell in vitro a recombinant expression vector that comprises: i) a first nucleotide sequence encoding one or more guide RNAs; and ii) a second nucleotide sequence encoding the fusion polypeptide (i.e., a fusion polypeptide comprising: i) a CRISPR-Cas effector polypeptide; and ii) two or more heterologous polypeptides, wherein one of the two or more heterologous polypeptides is an error-prone DNA polymerase and wherein one of the two or more heterologous polypeptides comprises an NES polypeptide). In some cases, the first nucleotide sequence is operably linked to a first promoter; and the second nucleotide sequence is operably linked to a second promoter.

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter: adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

Suitable expression vectors include viral expression vectors (e.g. viral vectors based on vaccinia virus: poliovirus: adenovirus: adeno-associated virus (AAV): SV40; herpes simplex virus: human immunodeficiency virus: a retroviral vector (e.g., Murine Leukemia Virus, spleen necrosis virus, and vectors derived from retroviruses such as Rous Sarcoma Virus. Harvey Sarcoma Virus, avian leukosis virus, a lentivirus, human immunodeficiency virus, myeloproliferative sarcoma virus, and mammary tumor virus); and the like. In some cases, a recombinant expression vector is a recombinant adeno-associated virus (AAV) vector. In some cases, a recombinant expression vector is a recombinant lentivirus vector. In some cases, a recombinant expression vector is a recombinant retroviral vector.

Suitable eukaryotic cells include in vitro cell lines, e.g., mammalian cell lines. Suitable mammalian cell lines include, but are not limited to. Hela cells (e.g., American Type Culture Collection (ATCC) No. CCL-2), CHO cells (e.g., ATCC Nos. CRL9618, CCL61, CRL9096), 293 cells (e.g., ATCC No. CRL-1573). Vero cells, NIH 3T3 cells (e.g., ATCC No. CRL-1658), Huh-7 cells. BHK cells (e.g., ATCC No. CCL10), PC12 cells (ATCC No. CRL1721). COS cells. COS-7 cells (ATCC No. CRL1651). RATI cells, mouse L cells (ATCC No. CCLI.3), human embryonic kidney (HEK) cells (ATCC No. CRL1573), HEK 293T cells, HLHepG2 cells, and the like.

Methods of introducing a nucleic acid (e.g., a recombinant expression vector) into a host cell are known in the art, and any convenient method can be used to introduce a nucleic acid into a cell. Suitable methods include e.g., viral infection, transfection, lipofection, electroporation, calcium phosphate precipitation, polyethyleneimine (PEI)-mediated transfection. DEAE-dextran mediated transfection, liposome-mediated transfection, particle gun technology: calcium phosphate precipitation, direct microinjection, nanoparticle-mediated nucleic acid delivery, and the like. A fusion polypeptide and a guide RNA can be present in a composition with a lipid. A fusion polypeptide and a guide RNA can be present in a lipid nanoparticle. Other suitable compositions are known in the art.

In some cases, a method of the present disclosure for modifying a target viral nucleic acid in the cytoplasm of a eukaryotic cell comprises: A) introducing into the eukaryotic cell gene editing components, wherein the gene editing components comprise: a) a fusion polypeptide comprising: i) a CRISPR-Cas effector polypeptide; and ii) two or more heterologous polypeptides, wherein one of the two or more heterologous polypeptides is an error-prone DNA polymerase and wherein one of the two or more heterologous polypeptides comprises an NES polypeptide; and b) one or more guide nucleic acids, wherein one or more guide nucleic acids comprise: i) a targeting region that comprises a nucleotide sequence that binds to a target sequence in the target viral nucleic acid; and ii) a protein-binding region that binds to the CRISPR-Cas effector polypeptide, thereby generating a modified eukaryotic cell; and B) infecting the modified eukaryotic cell with a virus comprising the target viral nucleic acid, wherein the target viral nucleic acid is contacted with the gene editing components, and wherein said contacting provides for modification of the target viral nucleic acid. In some cases, the infection step (step B) is carried out after the introduction step (step A). In some cases, step B is carried out from 2 hours to 96 hours after step A. For example, in some cases, step B is carried out from 2 hours to 4 hours, from 4 hours to 8 hours, from 8 hours to 12 hours, from 12 hours to 18 hours, from 18 hours to 24 hours, from 24 hours to 36 hours, from 36 hours to 48 hours, from 48 hours to 72 hours, or from 72 hours to 96 hours, after step A.

### CRISPR-Cas Effector Polypeptides

Suitable CRISPR-Cas effector polypeptides include Type II CRISPR-Cas effector polypeptides. Type III CRISPR Cas effector polypeptides. Type V CRISPR Cas effector polypeptides, and Type VI CRISPR-Cas effector polypeptides.

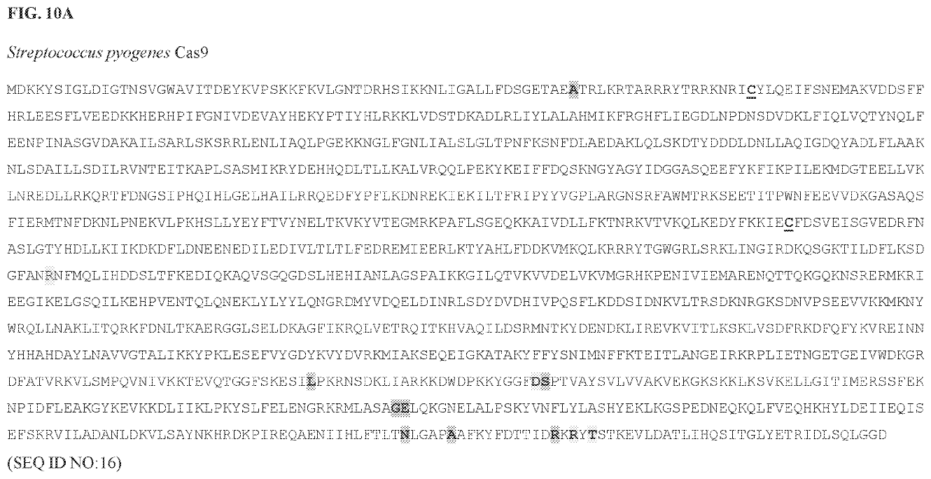

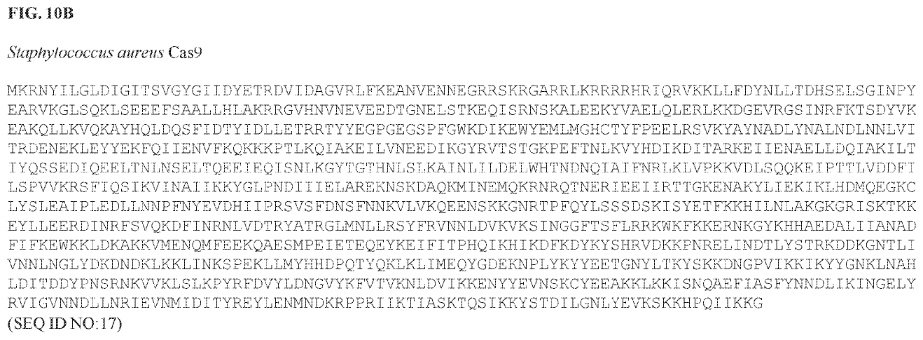

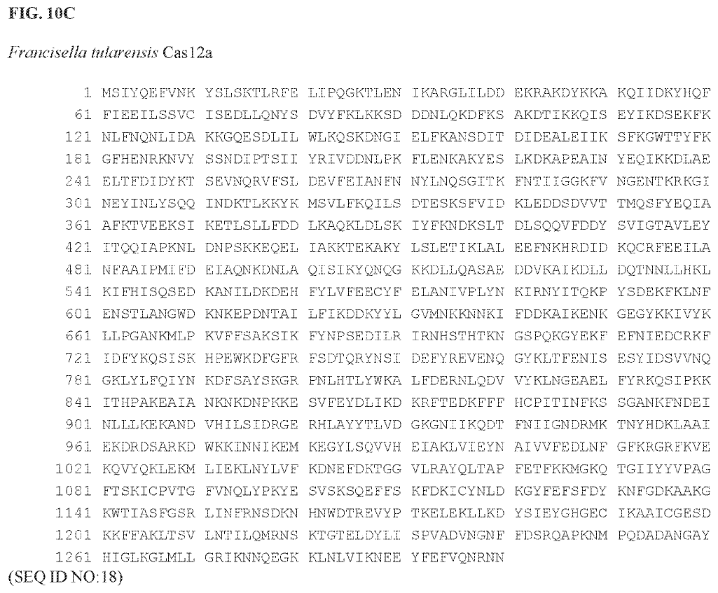

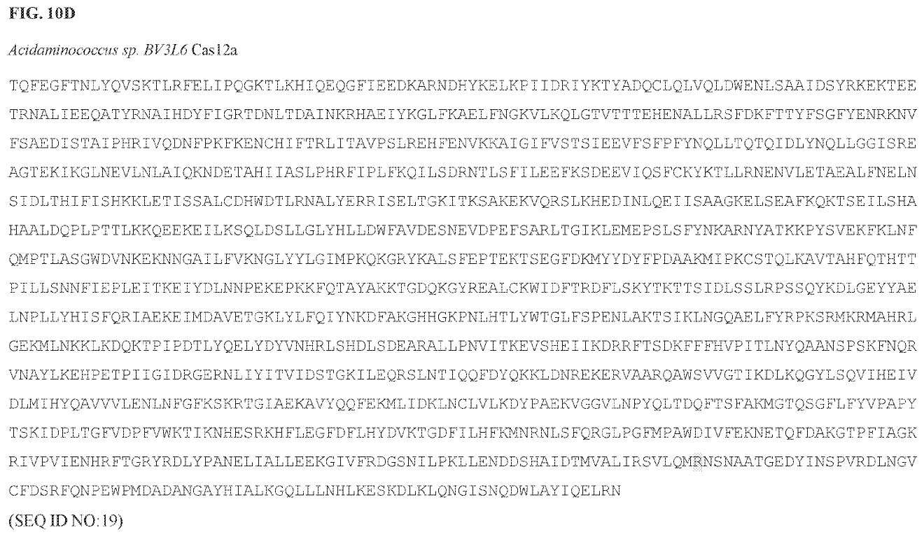

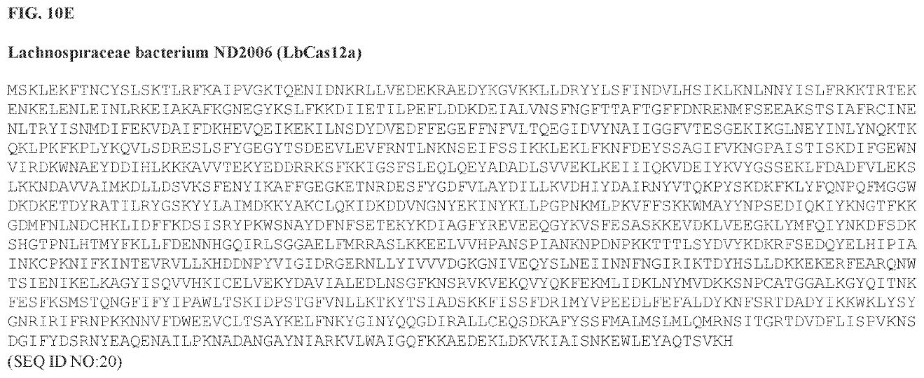

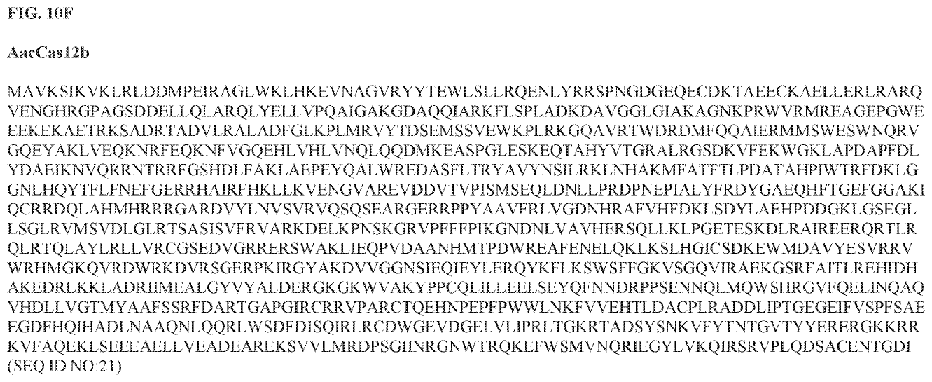

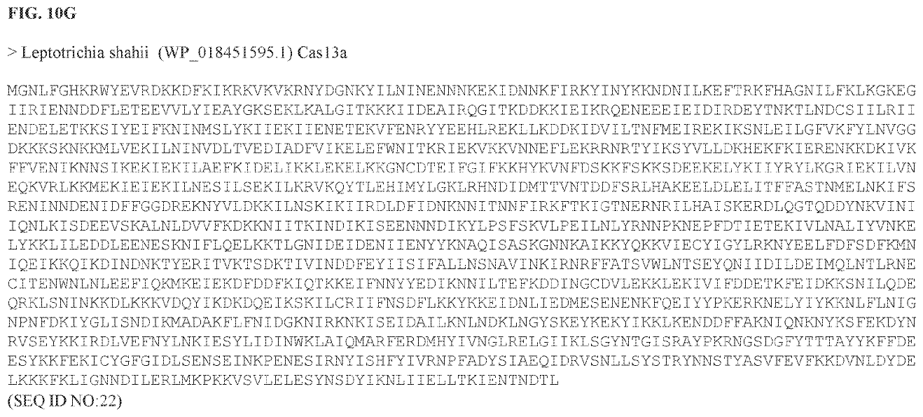

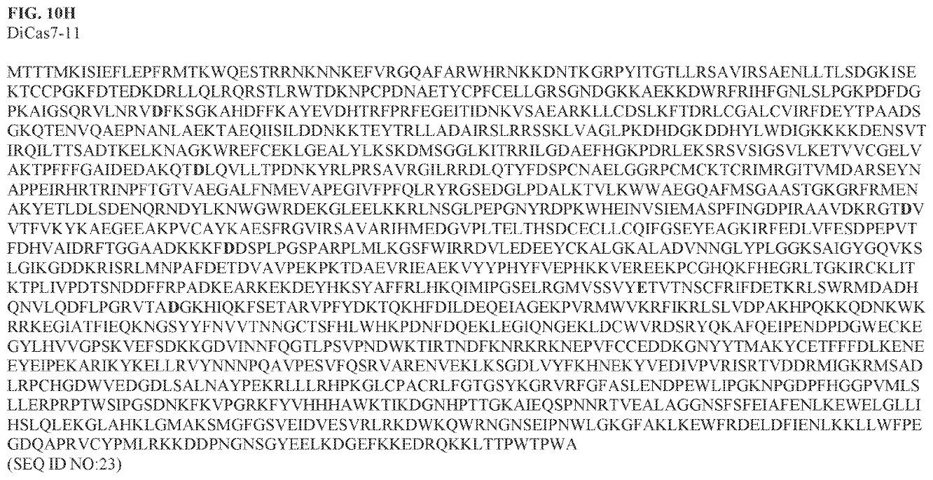

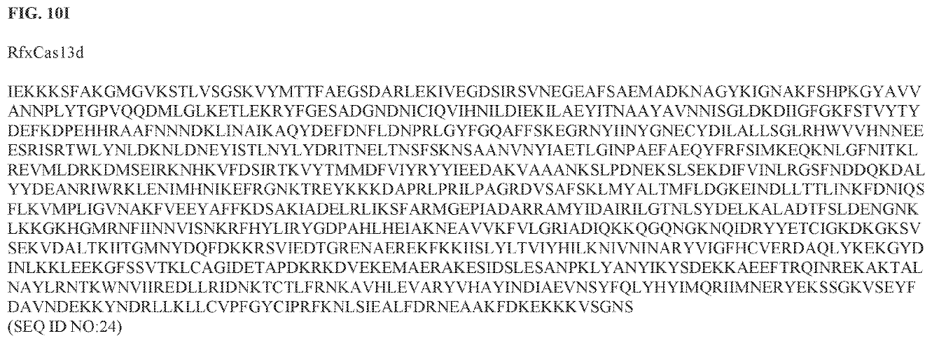

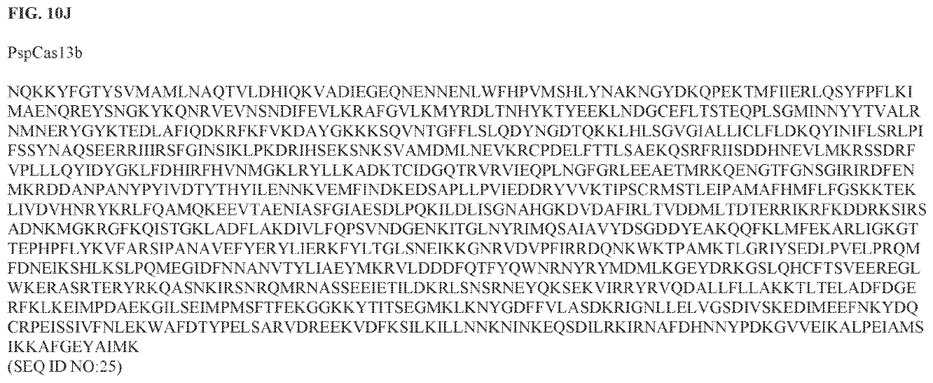

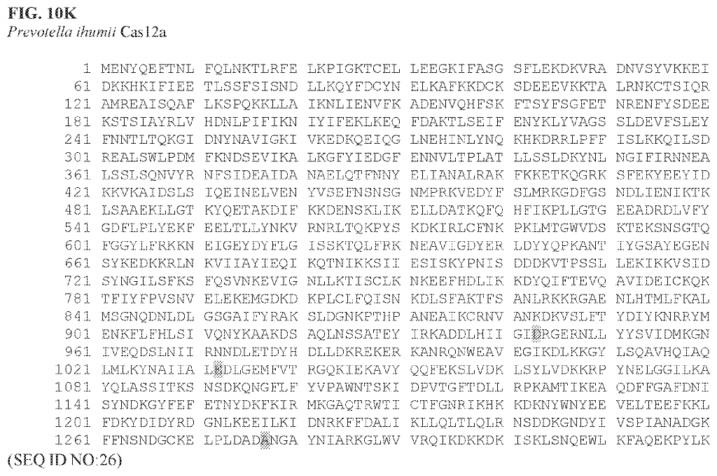

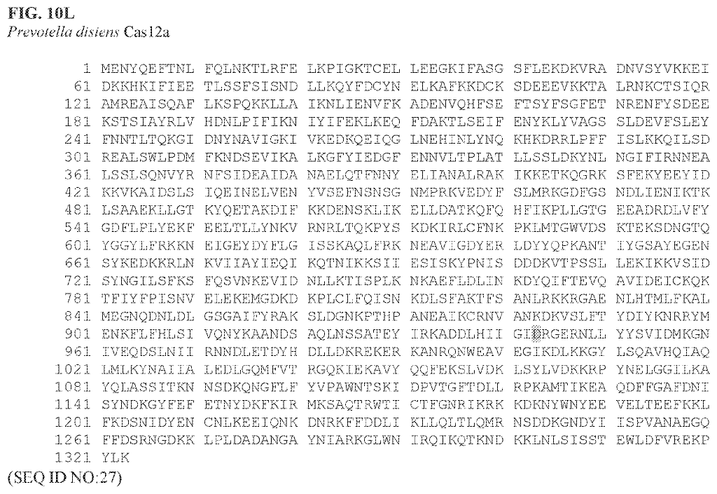

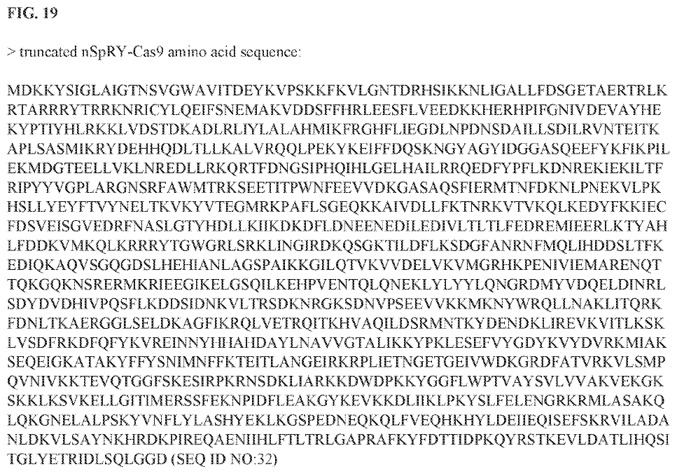

In some cases, the CRISPR-Cas effector polypeptide is a type II CRISPR-Cas effector polypeptide. In some cases, the type II CRISPR-Cas effector polypeptide is a Cas9 polypeptide, e.g., Staphylococcus aureus Cas9. Streptococcus pyogenes Cas9 (SpCas9), etc. In some cases, the CRISPR-Cas effector polypeptide is a variant of a wild-type SpCas9 and comprises one or more of the following substitutions: A61R, L1111R, A1322R, D1135L, S1136W, G1218K, E1219Q, N1317R, R1333P, R1335A, and T1337R. In some cases, the CRISPR-Cas effector polypeptide is an SpG polypeptide or a SpRY polypeptide: see, e.g., Walton et al. (2020) Science 368:290, and WO 2019/051097. SpRY is capable of targeting almost all protospacer-adjacent motifs (PAMs) (NRN>NYN). In some cases, the CRISPR-Cas effector polypeptide is a miniaturized nSpRY-Cas9 for which amino acids have been deleted. For example, a suitable CRISPR-Cas effector polypeptide is an SpCas9 polypeptide includes D1135V. R1135Q, and T1137R substitutions, relative to wild-type SpCas9. As another example, a suitable CRISPR-Cas effector polypeptide is an SpCas9 polypeptide includes D1135V, R1335Q, T1337R, and G1218R substitutions, relative to wild-type SpCas9. As another example, a suitable CRISPR-Cas effector polypeptide is an SpCas9 polypeptide includes D1135L, S1136W, G1218K, E1219Q, R1335A, and T1337R substitutions, relative to wild-type SpCas9. As another example, a suitable CRISPR-Cas effector polypeptide is an SpCas9 polypeptide includes L1111R, A1322R, D1135L, S1136W, G1218K, E1219Q, R1335A, and T1337R substitutions, relative to wild-type SpCas9. As another example, a suitable CRISPR-Cas effector polypeptide is an SpCas9 polypeptide includes A61R, L1111R, A1322R, D1135L, S1136W, G1218K, E1219Q, N1317R, R1333P, R1335A, and T1337R substitutions, relative to wild-type SpCas9. The amino acid sequence of a wild-type SpCas9 polypeptide is provided in FIG. 10A. As another example, a suitable CRISPR-Cas effector polypeptide comprises an amino acid sequence having at least 50%, at least 60%, at least 70%, at least 80%, at least 85%, at least 90%, at least 95%, at least 98%, or at least 99%, amino acid sequence identity to the amino acid sequence depicted in FIG. 19.

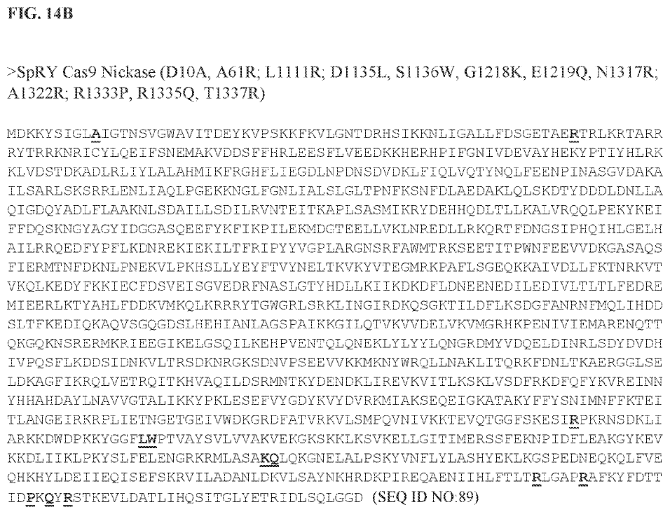

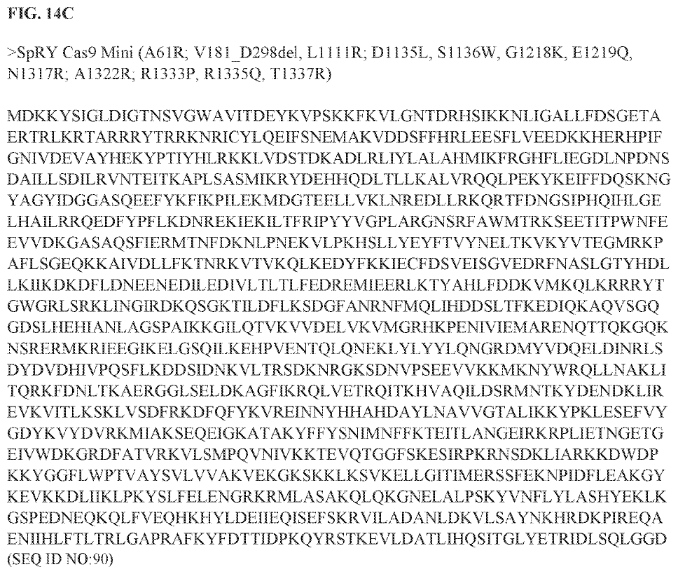

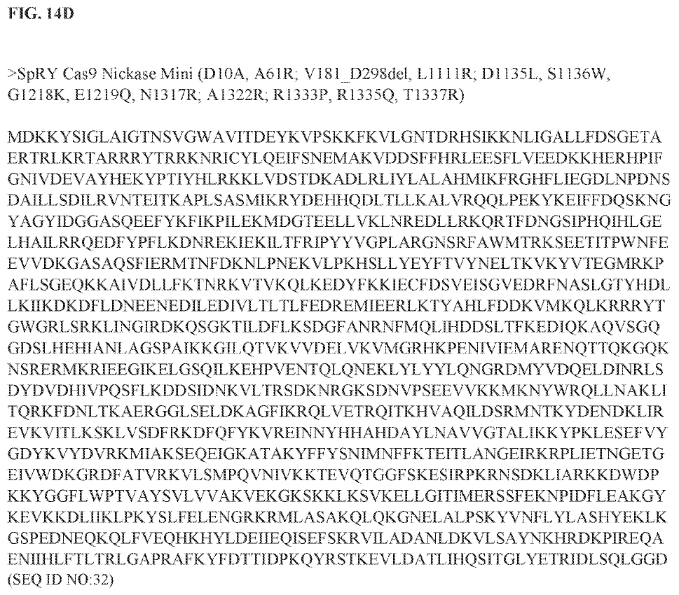

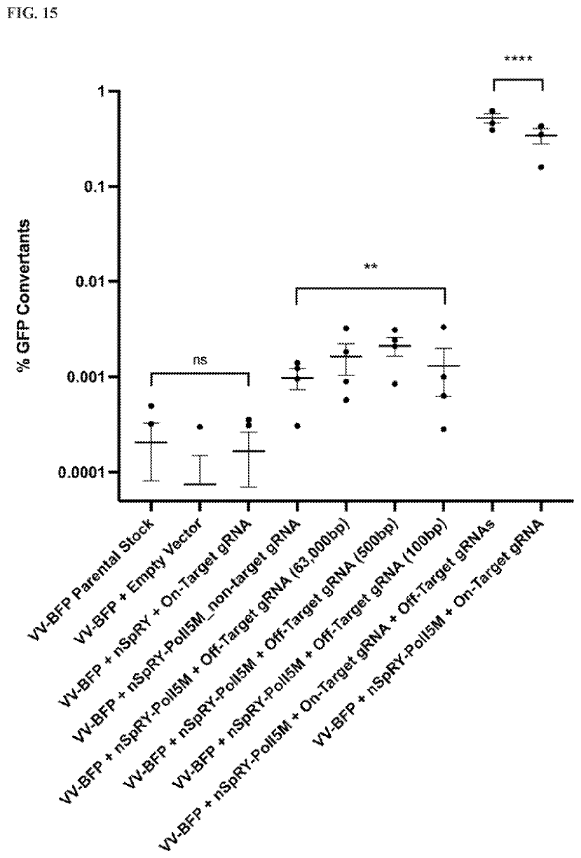

In some cases, the CRISPR-Cas effector polypeptide is a type V CRISPR-Cas effector polypeptide, e.g., a Cas12a, a Cas12b, a Cas12c, a Cas12d, or a Cas12e polypeptide. In some cases, the CRISPR-Cas effector polypeptide is a type VI CRISPR-Cas effector polypeptide, e.g., a Cas13a polypeptide, a Cas13b polypeptide, a Cas13c polypeptide, or a Cas13d polypeptide. In some cases, the CRISPR-Cas effector polypeptide is a Cas14 polypeptide. In some cases, the CRISPR-Cas effector polypeptide is a Cas14a polypeptide, a Cas14b polypeptide, or a Cas14c polypeptide. For example, a suitable CRISPR-Cas effector polypeptide comprises an amino acid sequence having at least 50%, at least 60%, at least 70%, at least 80%, at least 85%, at least 90%, at least 95%, at least 98%, or at least 99%, amino acid sequence identity to the amino acid sequence depicted in any one of FIG. 10A-10L. In some cases, a suitable CRISPR-Cas effector polypeptide comprises an amino acid sequence having at least 50%, at least 60%, at least 70%, at least 80%, at least 85%, at least 90%, at least 95%, at least 98%, or at least 99%, amino acid sequence identity to the amino acid sequence depicted in any one of FIG. 14A-14D.

In some cases, a CRISPR-Cas effector polypeptide suitable for use in a method, a system, or a composition of the present disclosure is a nickase CRISPR-Cas effector polypeptide, i.e., a CRISPR-Cas effector polypeptide that, when complexed with a guide RNA, binds to a target nucleic acid and cleaves only one strand of the target nucleic acid. For example, in some cases, a CRISPR-Cas effector polypeptide is a Spy Cas9 polypeptide comprising a D10A substitution.

### NESs and CPPs

In some cases, a heterologous polypeptide (a fusion partner) provides for subcellular localization, i.e., the heterologous polypeptide contains a subcellular localization sequence (e.g., a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some cases, a CRISPR-Cas effector fusion polypeptide does not include an NLS so that the protein is not targeted to the nucleus (which can be advantageous, e.g., when the target nucleic acid is present in the cytoplasm).

NESs are known in the art, and any NES can be used in a fusion polypeptide of the present disclosure. See, e.g., Xu et al. (2012) Mol. Biol. Cell 23:3677; and Fung et al. (2017) eLife 6:e23961. Examples of NESs include, e.g., LPPLERLTL (SEQ ID NO:34); LALKLAGLDL (SEQ ID NO:35); MEELSQALASSFSV (SEQ ID NO:36); EAETVSAMALLSVG (SEQ ID NO:37); ELDELMASLSDFKF (SEQ ID NO:38); VDQLRLERLQI (SEQ ID NO:39); IDLSGLTLQ (SEQ ID NO:40); LRALERLQID (SEQ ID NO:41); LQKKLEELEL (SEQ ID NO:42); MQELSNILNL (SEQ ID NO:43); LCQAFSDVIL (SEQ ID NO:44); RTFDMHSLESSLIDIMR (SEQ ID NO:45); TNLEALQKKLEELELDE (SEQ ID NO:46); RSFEMTEFNQALEEIKG (SEQ ID NO:47); PLQLPPLERLTL (SEQ ID NO:48); NELALKLAGLDI (SEQ ID NO:49); ERFEMFRELNEALEL (SEQ ID NO:50); DHAEKVAEKLEALSV (SEQ ID NO:51); QLVEELLKIICAFQL (SEQ ID NO:52); TNLEALQKKLEELEL (SEQ ID NO:53); DVKEEMTSALATMRV (SEQ ID NO:54); STNGSLAAEFRHLQL (SEQ ID NO:55); PSVQELTEQIHRLLM (SEQ ID NO:56); MNFKELKDFLKELNI (SEQ ID NO:57); ENFEILMKLKESLEL (SEQ ID NO:58); FETVYELTKMCTIRM (SEQ ID NO:59); SGKASSSLGLQDFDL (SEQ ID NO:60); PKYSDIDVDGLCSEL (SEQ ID NO:61); and VDLACTPTDVRDVDI (SEQ ID NO:62). An NES can have a length of from 8 amino acids to 25 amino acids. In some cases, the NES has the following amino acid sequence: LPPLERLTL (SEQ ID NO:34); and has a length of 9 amino acids.

In some cases, a fusion polypeptide comprises, in order from N-terminus to C-terminus: i) a CRISPR-Cas effector polypeptide; ii) one or more NESs; and iii) an error-prone DNA polymerase. In some cases, a fusion polypeptide comprises, in order from N-terminus to C-terminus: i) a CRISPR-Cas effector polypeptide; ii) an error-protein DNA polymerase; and iii) one or more NESs. In some cases, a fusion polypeptide comprises, in order from N-terminus to C-terminus: i) one or more NESs; ii) a CRISPR-Cas effector polypeptide; and iii) an error-prone DNA polymerase. In some cases, a fusion polypeptide comprises, in order from N-terminus to C-terminus: i) a first NES; ii) a CRISPR-Cas effector polypeptide; iii) an error-prone DNA polymerase; and iv) a second NES. In some cases, a fusion polypeptide comprises, in order from N-terminus to C-terminus: i) a first NES; ii) a CRISPR-Cas effector polypeptide; iii) a second NES; and iv) an error-prone DNA polymerase. A peptide linker can be interposed between any two polypeptides in a fusion protein, e.g.: i) between an NES and a CRISPR-Cas effector polypeptide; ii) between a first NES and a second NES; iii) between a CRISPR-Cas effector polypeptide and an error-prone DNA polymerase; iv) between an error-prone DNA polymerase and an NES; and the like.

In some cases, the heterologous polypeptide can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6XHis tag; a hemagglutinin (HA) tag; a FLAG tag; a Myc tag; and the like).

In some cases, a CRISPR-Cas effector fusion polypeptide includes a “Protein Transduction Domain” or PTD (also known as a CPP—cell penetrating peptide), which refers to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus of a fusion polypeptide. In some embodiments, a PTD is covalently linked to the carboxyl terminus of a fusion polypeptide. In some cases, the PTD is inserted internally in a fusion polypeptide at a suitable insertion site. Examples of PTDs include but are not limited to a minimal undecapeptide protein transduction domain (corresponding to residues 47-57 of HIV-1 TAT comprising YGRKKRRQRRR; SEQ ID NO:63); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:64); Transportan GWTLNSAGYLLGKINLKALAALAKKIL (SEQ ID NO:65); KALAWEAKLAKALAKALAKHLAKALAKALKCEA (SEQ ID NO:66); and RQIKIWFQNRRMKWKK (SEQ ID NO:67). Exemplary PTDs include but are not limited to, YGRKKRRQRRR (SEQ ID NO:63), RKKRRQRRR (SEQ ID NO:68); an arginine homopolymer of from 3 arginine residues to 50 arginine residues; Exemplary PTD domain amino acid sequences include, but are not limited to, any of the following: YGRKKRRQRRR (SEQ ID NO:63); RKKRRQRR (SEQ ID NO:69); YARAAARQARA (SEQ ID NO:70); THRLPRRRRRR (SEQ ID NO:71); and GGRRARRRRRR (SEQ ID NO:72). In some cases, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

### Error-Prone DNA Polymerases

A number of error-prone DNA polymerases are known in the art, and any known error-prone DNA polymerase is suitable for use in a fusion polypeptide of the present disclosure. A suitable error-prone DNA polymerase possesses nick translating activity.

Suitable error-prone DNA polymerases include, but are not limited to, Taq polymerase, Thermus flavus DNA polymerase I, Thermus thermophilus HB-8 DNA polymerase I, Thermophilus ruber DNA polymerase I, Thermophilus brokianus DNA polymerase I, Thermophilus caldophilus GK14 DNA polymerase I, Thermophilus filoformis DNA polymerase I, Bacillus stearothermophilus DNA polymerase I, Bacillus caldotonex YT-G DNA polymerase I, and Bacillus caldovelox YT-F DNA polymerase I. Suitable error-prone DNA polymerases include, but are not limited to, a Niastella koreensis error-prone DNA polymerase, a Mucilaginibacter paludis error-prone DNA polymerase, a Methylobacterium extorquens error-prone DNA polymerase, and a Stenotrophomonas maltophilia error-prone DNA polymerase.

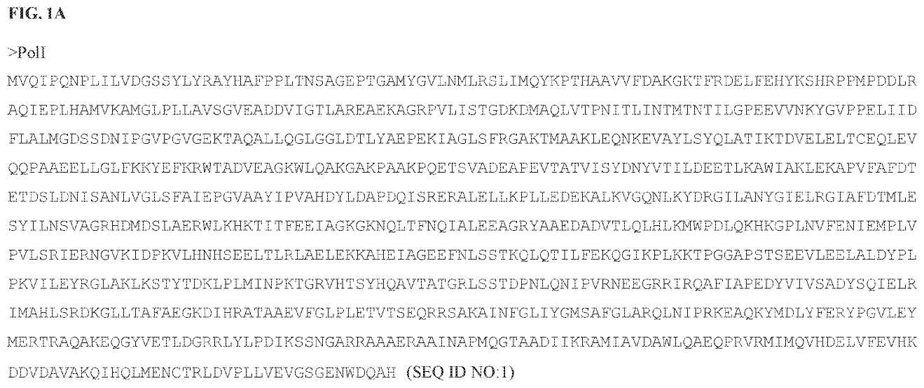

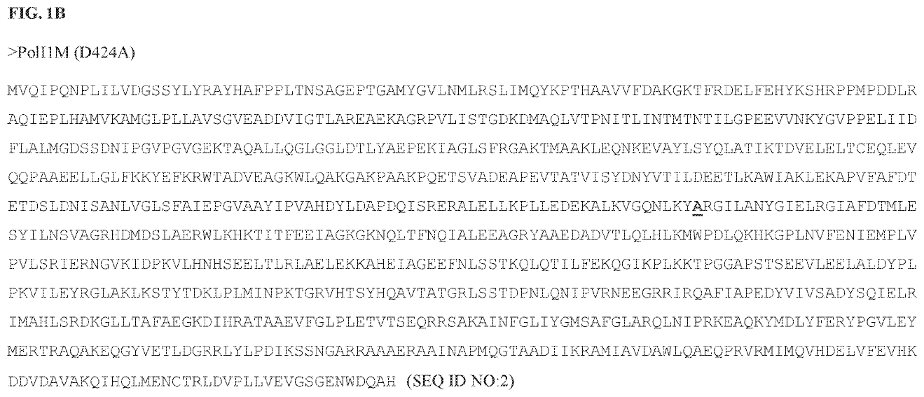

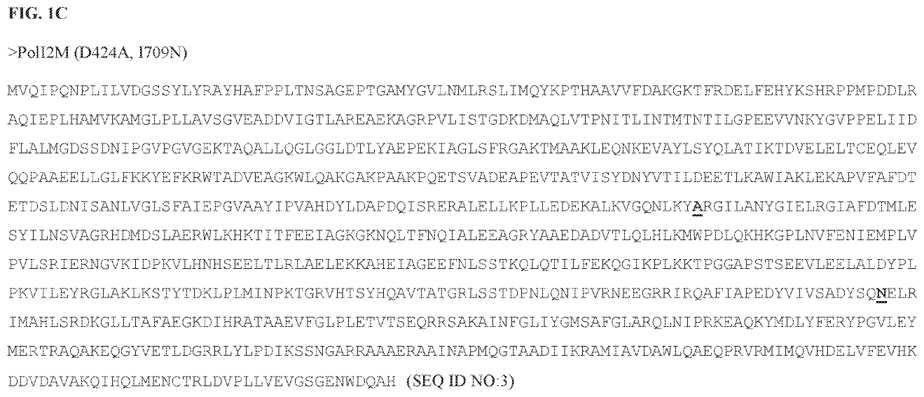

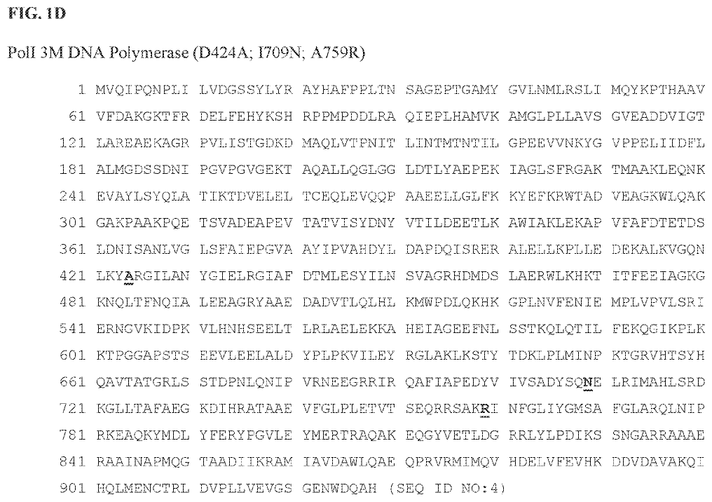

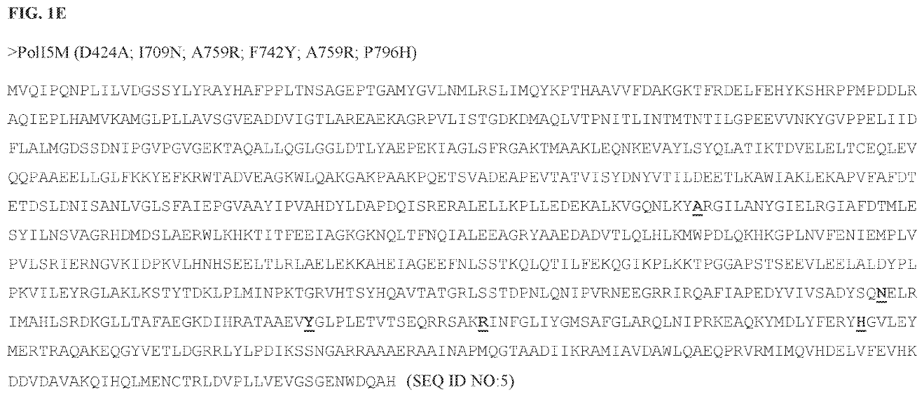

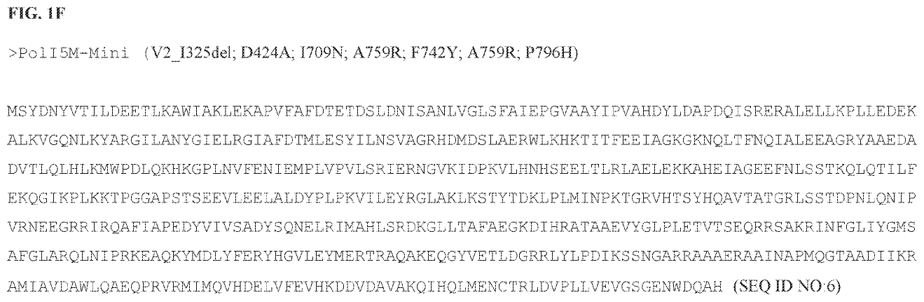

In some cases, a suitable error-prone DNA polymerase comprises an amino acid sequence having at least 85%, at least 90%, at least 95%, at least 98%, at least 99%, or 100%, amino acid sequence identity to the DNA polymerase I amino acid sequence depicted in any one of FIG. 1A-1F.

In some cases, a suitable error-prone DNA polymerase is Escherichia coli DNA polymerase I, with three fidelity-reducing mutations; this error-prone DNA polymerase is referred to as Poll3M. Poll3M comprises D424A, I709N, and A759R substitutions relative to wild-type E. coli DNA polymerase I. In some cases, a suitable error-prone DNA polymerase comprises an amino acid sequence having at least 85%, at least 90%, at least 95%, at least 98%, at least 99%, or 100%, amino acid sequence identity to the DNA polymerase I amino acid sequence depicted in FIG. 1A; where the DNA polymerase has an Ala at amino acid position 424, an Asn at amino acid position 709, and an Arg at amino acid position 759 of the amino acid sequence depicted in FIG. 1A, or a corresponding amino acid in another DNA polymerase.

In some cases, a suitable error-prone DNA polymerase is Escherichia coli DNA polymerase I, with five fidelity-reducing mutations: D424 A, I709N, A759R, F742Y, and P796H. In some cases, a suitable error-prone DNA polymerase comprises an amino acid sequence having at least 85%, at least 90%, at least 95%, at least 98%, at least 99%, or 100%, amino acid sequence identity to the DNA polymerase I amino acid sequence depicted in FIG. 1A; where the DNA polymerase has an Ala at amino acid position 424, an Asn at amino acid position 709, an Arg at amino acid position 759, a Tyr at amino acid position 742, and a His at amino acid position 796; or corresponding amino acids in another DNA polymerase.

In some cases, a suitable error-prone DNA polymerase comprises an amino acid sequence having at least 85%, at least 90%, at least 95%, at least 98%, or at least 99%, amino acid sequence identity to the DNA polymerase I amino acid sequence depicted in FIG. 1A; where the DNA polymerase has an Ala at amino acid position 424, and an Asn at amino acid position 709.

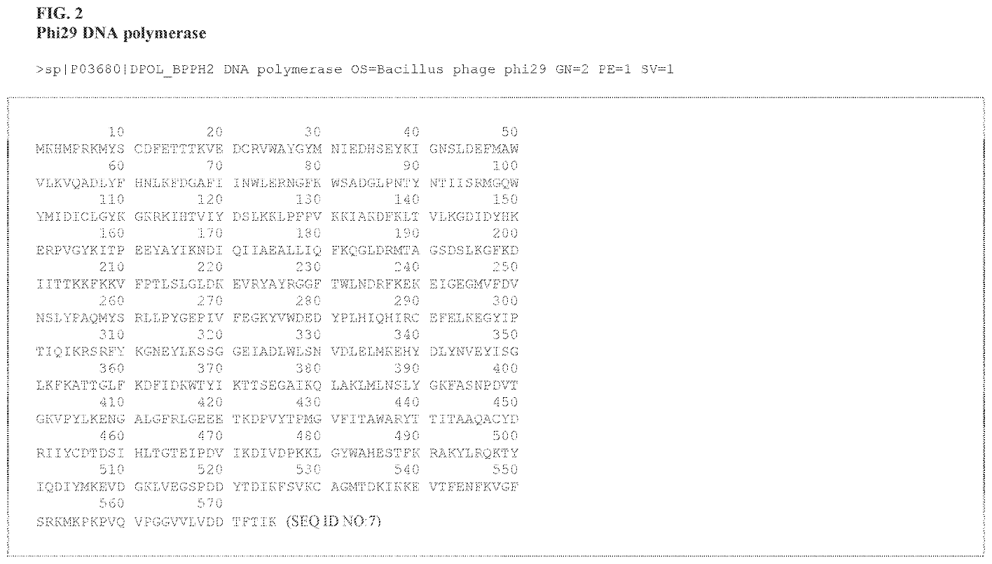

In some cases, a suitable error-prone DNA polymerase comprises an amino acid sequence having at least 85%, at least 90%, at least 95%, at least 98%, at least 99%, or 100%, amino acid sequence identity to the DNA polymerase I amino acid sequence depicted in FIG. 2.

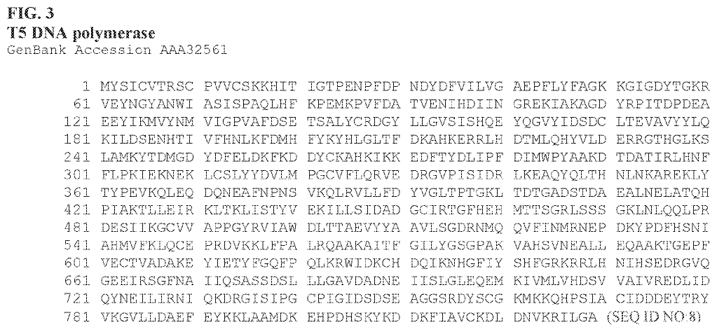

In some cases, a suitable error-prone DNA polymerase comprises an amino acid sequence having at least 85%, at least 90%, at least 95%, at least 98%, at least 99%, or 100%, amino acid sequence identity to the DNA polymerase I amino acid sequence depicted in FIG. 3.

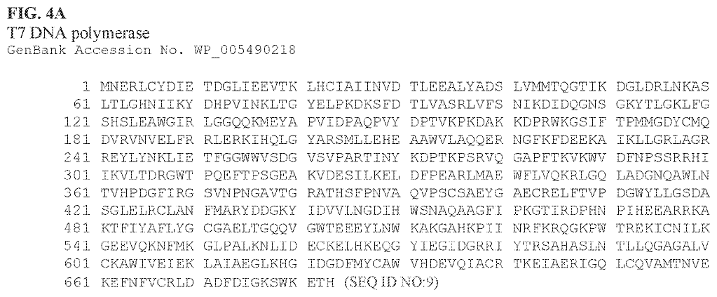

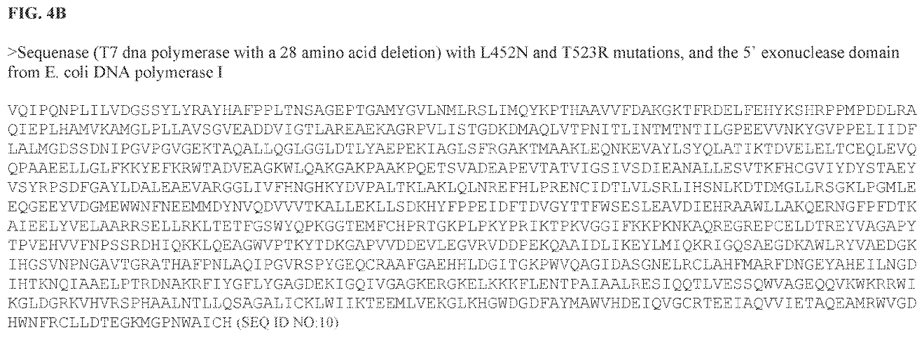

In some cases, a suitable error-prone DNA polymerase comprises an amino acid sequence having at least 85%, at least 90%, at least 95%, at least 98%, at least 99%, or 100%, amino acid sequence identity to the DNA polymerase I amino acid sequence depicted in FIG. 4A or FIG. 4B.

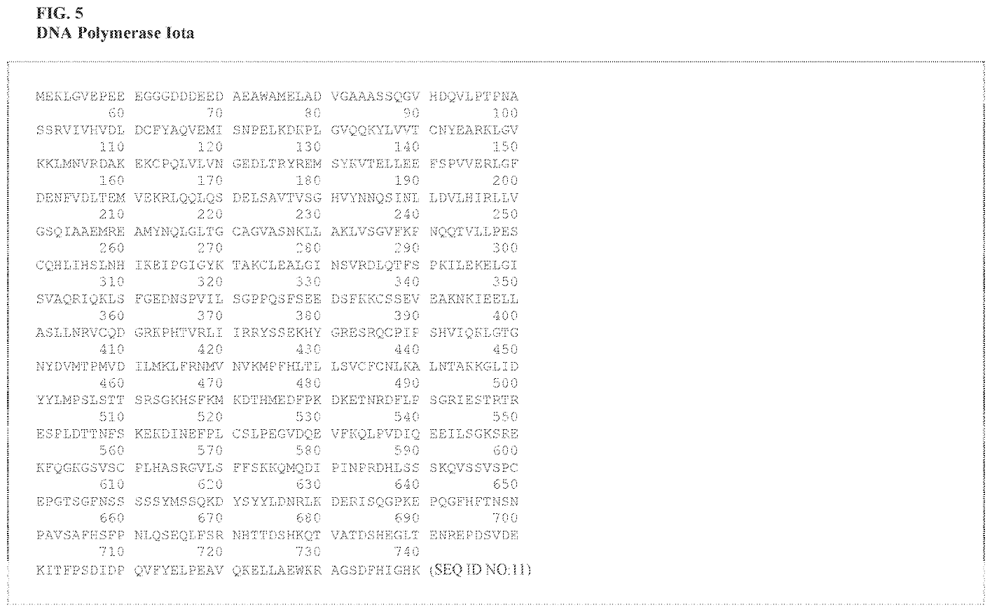

In some cases, a suitable error-prone DNA polymerase comprises an amino acid sequence having at least 85%, at least 90%, at least 95%, at least 98%, at least 99%, or 100%, amino acid sequence identity to the DNA polymerase iota amino acid sequence depicted in FIG. 5.

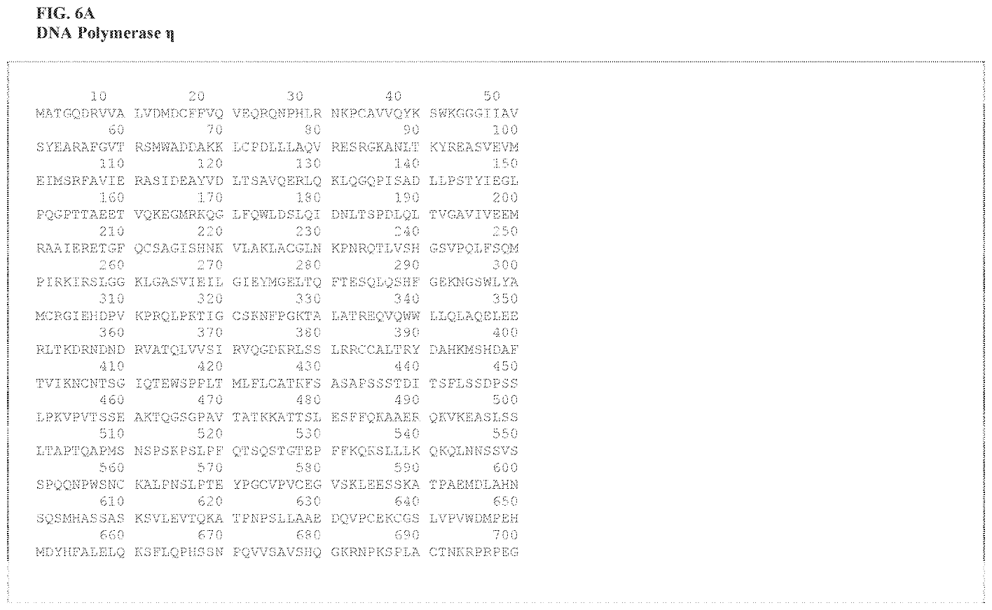

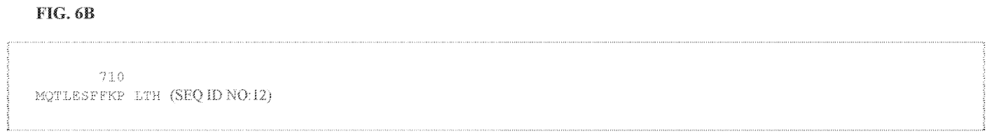

In some cases, a suitable error-prone DNA polymerase comprises an amino acid sequence having at least 85%, at least 90%, at least 95%, at least 98%, at least 99%, or 100%, amino acid sequence identity to the DNA polymerase η amino acid sequence depicted in FIG. 6A-6B.

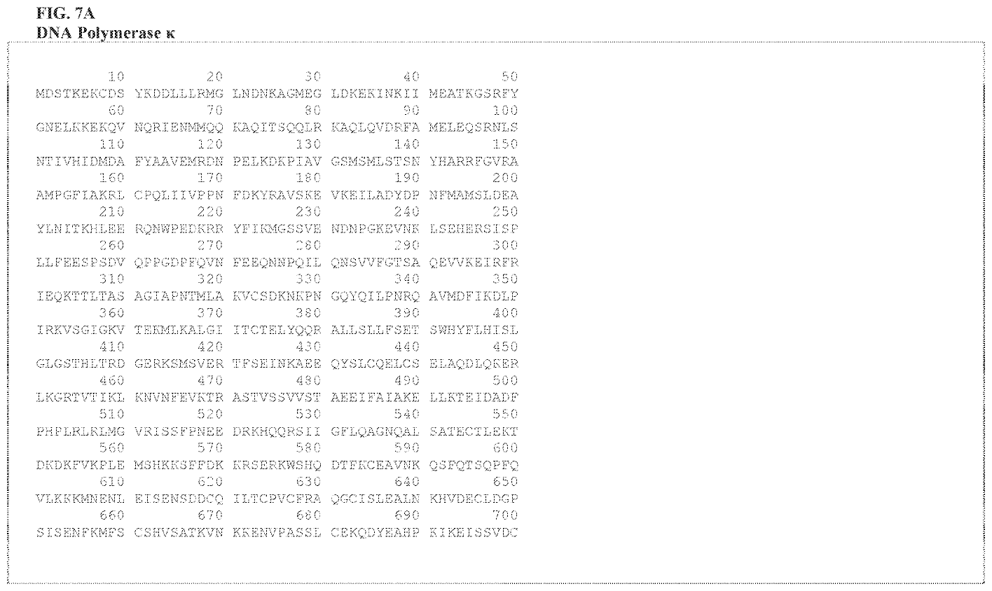

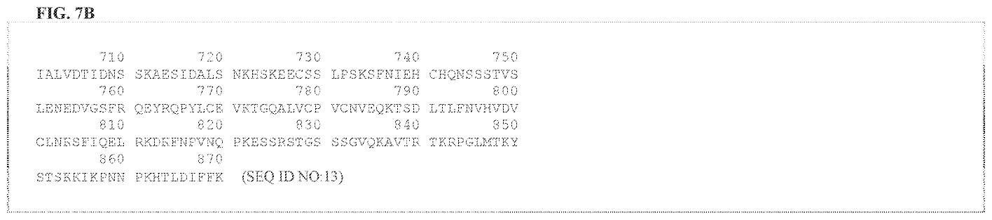

In some cases, a suitable error-prone DNA polymerase comprises an amino acid sequence having at least 85%, at least 90%, at least 95%, at least 98%, at least 99%, or 100%, amino acid sequence identity to the DNA polymerase κ amino acid sequence depicted in FIG. 7A-7B.

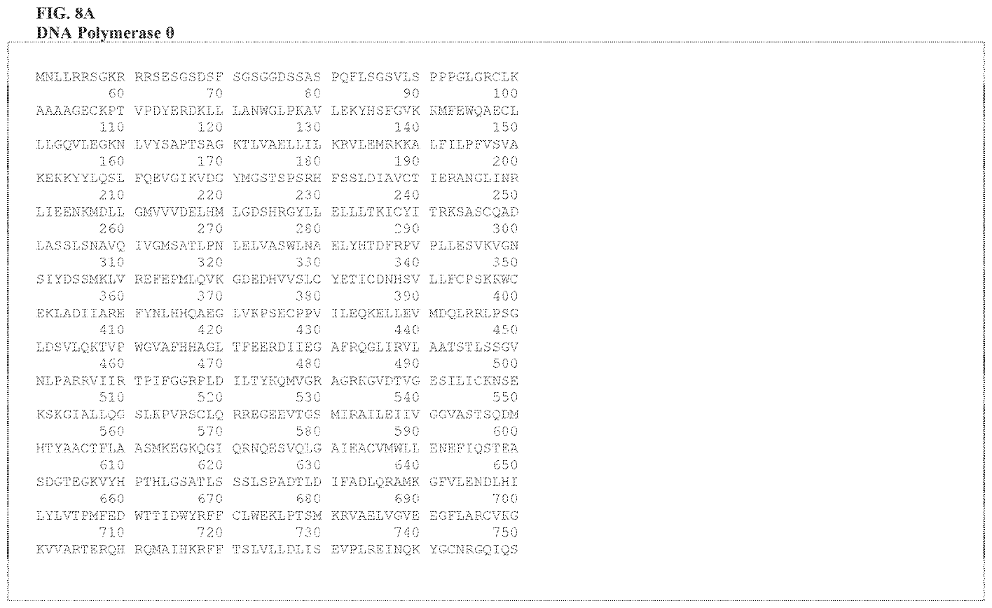

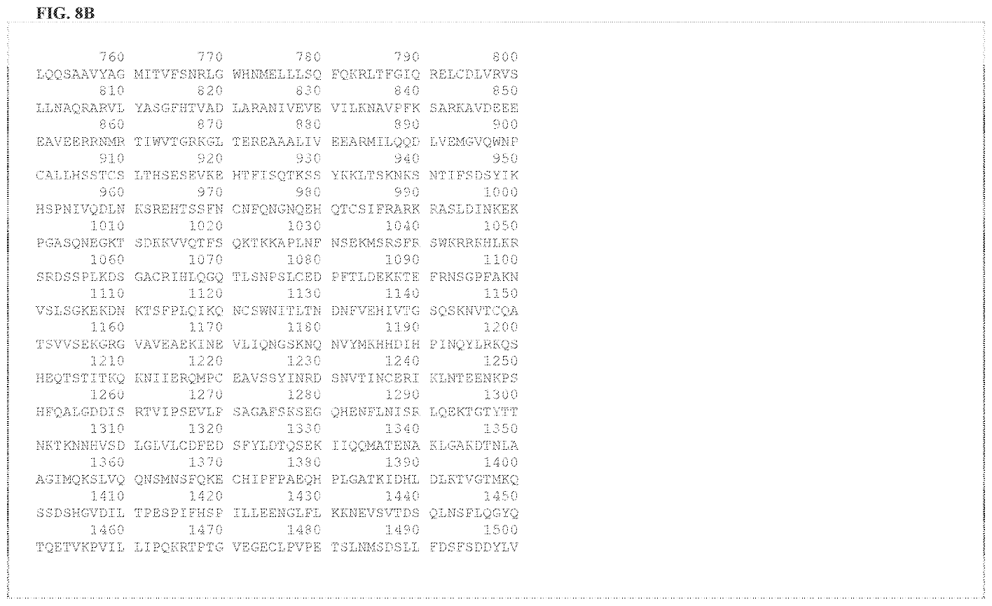

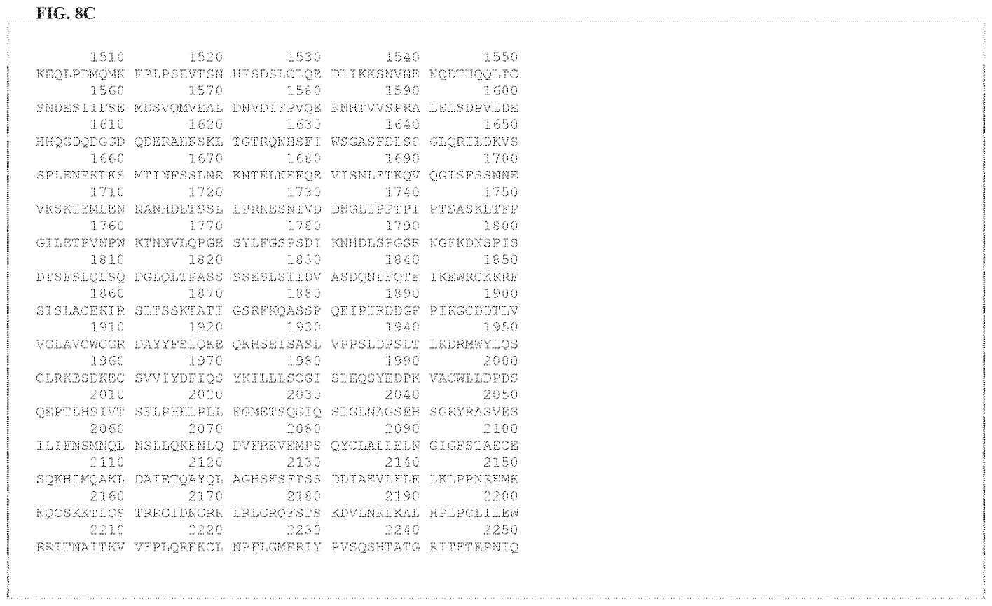

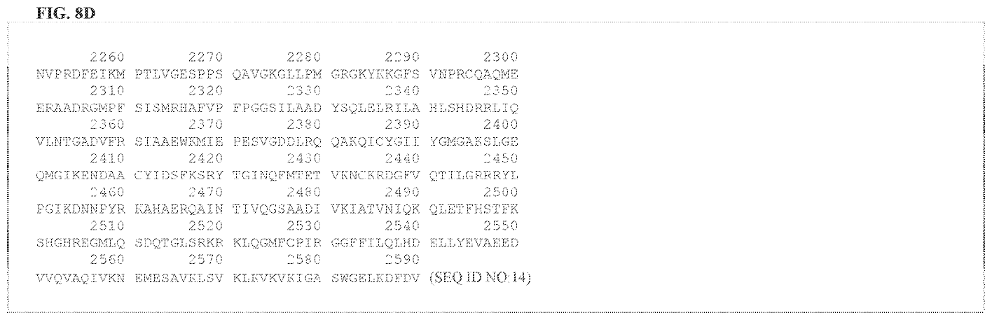

In some cases, a suitable error-prone DNA polymerase comprises an amino acid sequence having at least 85%, at least 90%, at least 95%, at least 98%, at least 99%, or 100%, amino acid sequence identity to the DNA polymerase θ amino acid sequence depicted in FIG. 8A-8D.

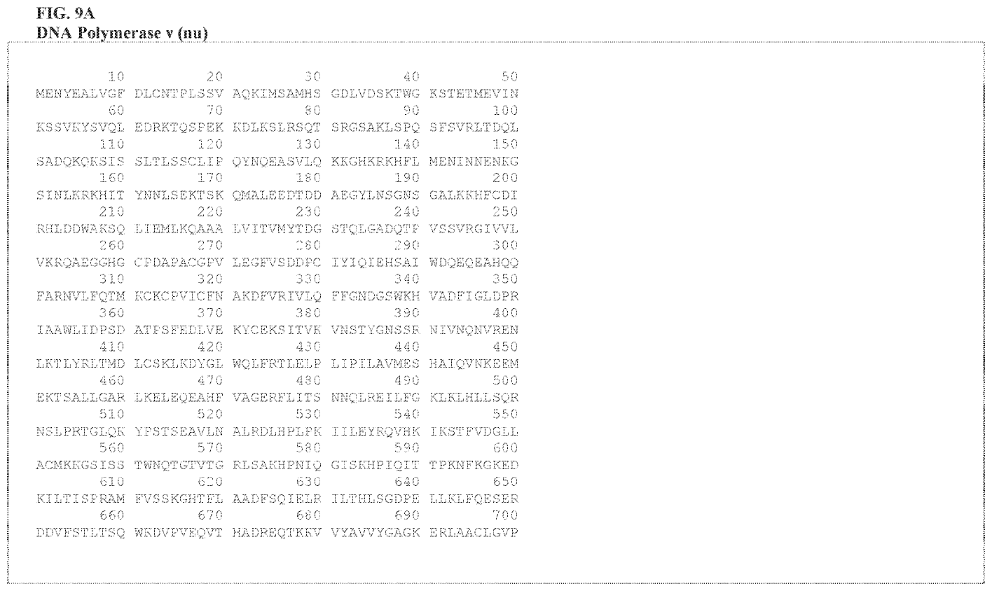

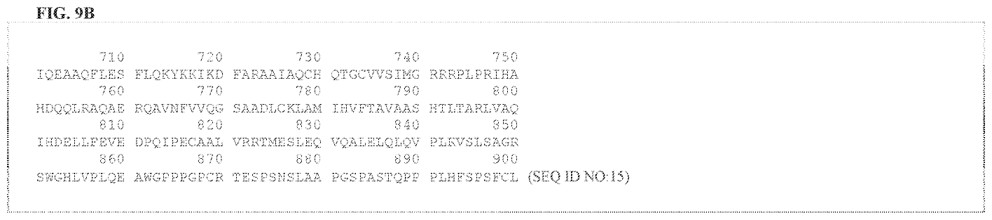

In some cases, a suitable error-prone DNA polymerase comprises an amino acid sequence having at least 85%, at least 90%, at least 95%, at least 98%, at least 99%, or 100%, amino acid sequence identity to the DNA polymerase v (nu) amino acid sequence depicted in FIG. 9A-9B.

In some cases, a suitable error-prone DNA polymerase comprises an amino acid sequence having at least 85%, at least 90%, at least 95%, at least 98%, at least 99%, or 100%, amino acid sequence identity to a DNA polymerase β having the following amino acid sequence:

(SEQ ID NO: 73)
MSKRKAPQETLNGGITDMLTELANFEKNVSQAIHKYNAYRKAASVIAKYPHKIKSGAEAKKLPG
VGTKIAEKIDEFLATGKLRKLEKIRQDDTSSSINFLTRVSGIGPSAARKFVDEGIKTLEDLRKNEDK
LNHHQRIGLKYFGDFEKRIPREEMLQMQDIVLNEVKKVDSEYIATVCGSFRRGAESSGDMDVLLT
HPSFTSESTKQPKLLHQVVEQLQKVHFITDTLSKGETKFMGVCQLPSKNDEKEYPHRRIDIRLIPK
DQYYCGVLYFTGSDIFNKNMRAHALEKGFTINEYTIRPLGVTGVAGEPLPVDSEKDIFDYIQWKY
REPKDRSE.

In some cases, a suitable error-prone DNA polymerase comprises an amino acid sequence having at least 85%, at least 90%, at least 95%, at least 98%, at least 99%, or 100%, amino acid sequence identity to a DNA polymerase iota having the following amino acid sequence:

(SEQ ID NO: 11)
MEKLGVEPEEEGGGDDDEEDAEAWAMELADVGAAASSQGVHDQVLPTPNASSRVIVHVDLDCF
YAQVEMISNPELKDKPLGVQQKYLVVTCNYEARKLGVKKLMNVRDAKEKCPQLVLVNGEDLTR
YREMSYKVTELLEEFSPVVERLGFDENFVDLTEMVEKRLQQLQSDELSAVTVSGHVYNNQSINLL
DVLHIRLLVGSQIAAEMREAMYNQLGLTGCAGVASNKLLAKLVSGVFKPNQQTVLLPESCQHLIH
SLNHIKEIPGIGYKTAKCLEALGINSVRDLQTFSPKILEKELGISVAQRIQKLSFGEDNSPVILSGPPQ
SFSEEDSFKKCSSEVEAKNKIEELLASLLNRVCQDGRKPHTVRLIIRRYSSEKHYGRESRQCPIPSH
VIQKLGTGNYDVMTPMVDILMKLFRNMVNVKMPFHLTLLSVCFCNLKALNTAKKGLIDYYLMP
SLSTTSRSGKHSFKMKDTHMEDFPKDKETNRDFLPSGRIESTRTRESPLDTTNFSKEKDINEFPLCS
LPEGVDQEVFKQLPVDIQEEILSGKSREKFQGKGSVSCPLHASRGVLSFFSKKQMQDIPINPRDHL
SSSKQVSSVSPCEPGTSGFNSSSSSYMSSQKDYSYYLDNRLKDERISQGPKEPQGFHFTNSNPAVS
AFHSFPNLQSEQLFSRNHTTDSHKQTVATDSHEGLTENREPDSVDEKITFPSDIDPQVFYELPEAVQ
KELLAEWKRAGSDFHIGHK. In some cases, such a DNA polymerase generates T →
G substitutions.

In some cases, a suitable error-prone DNA polymerase comprises an amino acid sequence having at least 85%, at least 90%, at least 95%, at least 98%, at least 99%, or 100%, amino acid sequence identity to a DNA polymerase iota having the following amino acid sequence (amino acids 1-445 of DNA polymerase iota):

(SEQ ID NO: 74)
MEKLGVEPEEEGGGDDDEEDAEAWAMELADVGAAASSQGVHDQVLPTPNASSRVIVHVDLDCF
YAQVEMISNPELKDKPLGVQQKYLVVTCNYEARKLGVKKLMNVRDAKEKCPQLVLVNGEDLTR
YREMSYKVTELLEEFSPVVERLGFDENFVDLTEMVEKRLQQLQSDELSAVTVSGHVYNNQSINLL
DVLHIRLLVGSQIAAEMREAMYNQLGLTGCAGVASNKLLAKLVSGVFKPNQQTVLLPESCQHLIH
SLNHIKEIPGIGYKTAKCLEALGINSVRDLQTFSPKILEKELGISVAQRIQKLSFGEDNSPVILSGPPQ
SFSEEDSFKKCSSEVEAKNKIEELLASLLNRVCQDGRKPHTVRLIIRRYSSEKHYGRESRQCPIPSH
VIQKLGTGNYDVMTPMVDILMKLFRNMVNVKMPFHLTLLSVCFCNLKALNTAK; and having a
length of 445 amino acids. In some cases, such a DNA polymerase
generates T → G substitutions. In some cases, such a DNA polymerase
has a T → G error rate approaching 1.

In some cases, a suitable error-prone DNA polymerase comprises an amino acid sequence having at least 85%, at least 90%, at least 95%, at least 98%, at least 99%, or 100%, amino acid sequence identity to a DNA polymerase iota having the following amino acid sequence (amino acids 26-445 of DNA polymerase iota):

(SEQ ID NO: 75)
ELADVGAAASSQGVHDQVLPTPNASSRVIVHVDLDCFYAQVEMISNPELKDKPLGVQQKYLVVT
CNYEARKLGVKKLMNVRDAKEKCPQLVLVNGEDLTRYREMSYKVTELLEEFSPVVERLGFDEN
FVDLTEMVEKRLQQLQSDELSAVTVSGHVYNNQSINLLDVLHIRLLVGSQIAAEMREAMYNQLG
LTGCAGVASNKLLAKLVSGVFKPNQQTVLLPESCQHLIHSLNHIKEIPGIGYKTAKCLEALGINSV
RDLQTFSPKILEKELGISVAQRIQKLSFGEDNSPVILSGPPQSFSEEDSFKKCSSEVEAKNKIEELLAS
LLNRVCQDGRKPHTVRLIIRRYSSEKHYGRESRQCPIPSHVIQKLGTGNYDVMTPMVDILMKLFR
NMVNVKMPFHLTLLSVCFCNLKALNTAK; and having a length of 419 amino acids.
In some cases, such a DNA polymerase generates T → G substitutions. In
some cases, such a DNApolymerase has a T → G error rate approaching 1.

In some cases, a suitable error-prone DNA polymerase comprises an amino acid sequence having at least 85%, at least 90%, at least 95%, at least 98%, at least 99%, or 100%, amino acid sequence identity to a DNA polymerase nu (v) having the following amino acid sequence:

(SEQ ID NO: 76)
ENYEALVGFDLCNTPLSSVAQKIMSAMHSGDLVDSKTWGKSTETMEVINKSSVKYSVQLEDRKT
QSPEKKDLKSLRSQTSRGSAKLSPQSFSVRLTDQLSADQKQKSISSLTLSSCLIPQYNQEASVLQKK
GHKRKHFLMENINNENKGSINLKRKHITYNNLSEKTSKQMALEEDTDDAEGYLNSGNSGALKK
HFCDIRHLDDWAKSQLIEMLKQAAALVITVMYTDGSTQLGADQTPVSSVRGIVVLVKRQAEGGH
GCPDAPACGPVLEGFVSDDPCIYIQIEHSAIWDQEQEAHQQFARNVLFQTMKCKCPVICFNAKDF
VRIVLQFFGNDGSWKHVADFIGLDPRIAAWLIDPSDATPSFEDLVEKYCEKSITVKVNSTYGNSSR
NIVNQNVRENLKTLYRLTMDLCSKLKDYGLWQLFRTLELPLIPILAVMESHAIQVNKEEMEKTSA
LLGARLKELEQEAHFVAGERFLITSNNQLREILFGKLKLHLLSQRNSLPRTGLQKYPSTSEAVLNA
LRDLHPLPKIILEYRQVHKIKSTFVDGLLACMKKGSISSTWNQTGTVTGRLSAKHPNIQGISKHPI
QITTPKNFKGKEDKILTISPRAMFVSSKGHTFLAADFSQIELRILTHLSGDPELLKLFQESERDDVFS
TLTSQWKDVPVEQVTHADREQTKKVVYAVVYGAGKERLAACLGVPIQEAAQFLESFLQKYKKI
KDFARAAIAQCHQTGCVVSIMGRRRPLPRIHAHDQQLRAQAERQAVNFVVQGSAADLCKLAMI
HVFTAVAASHTLTARLVAQIHDELLFEVEDPQIPECAALVRRTMESLEQVQALELQLQVPLKVSLS
AGRSWGHLVPLQEAWGPPPGPCRTESPSNSLAAPGSPASTQPPPLHFSPSFCL. In
some cases, such a DNA polymerase generates G → T substitutions.

In some cases, a suitable error-prone DNA polymerase comprises an amino acid sequence having at least 85%, at least 90%, at least 95%, at least 98%, at least 99%, or 100%, amino acid sequence identity to a DNA polymerase eta (η) having the following amino acid sequence:

(SEQ ID NO: 12)
MATGQDRVVALVDMDCFFVQVEQRQNPHLRNKPCAVVQYKSWKGGGIIAVSYEARAFGVTRSM
WADDAKKLCPDLLLAQVRESRGKANLTKYREASVEVMEIMSRFAVIERASIDEAYVDLTSAVQER
LQKLQGQPISADLLPSTYIEGLPQGPTTAEETVQKEGMRKQGLFQWLDSLQIDNLTSPDLQLTVG
AVIVEEMRAAIERETGFQCSAGISHNKVLAKLACGLNKPNRQTLVSHGSVPQLFSQMPIRKIRSLG
GKLGASVIEILGIEYMGELTQFTESQLQSHFGEKNGSWLYAMCRGIEHDPVKPRQLPKTIGCSKNF
PGKTALATREQVQWWLLQLAQELEERLTKDRNDNDRVATQLVVSIRVQGDKRLSSLRRCCALTR
YDAHKMSHDAFTVIKNCNTSGIQTEWSPPLTMLFLCATKFSASAPSSSTDITSFLSSDPSSLPKVPV
TSSEAKTQGSGPAVTATKKATTSLESFFQKAAERQKVKEASLSSLTAPTQAPMSNSPSKPSLPFQTS
QSTGTEPFFKQKSLLLKQKQLNNSSVSSPQQNPWSNCKALPNSLPTEYPGCVPVCEGVSKLEESS
KATPAEMDLAHNSQSMHASSASKSVLEVTQKATPNPSLLAAEDQVPCEKCGSLVPVWDMPEHM
DYHFALELQKSFLQPHSSNPQVVSAVSHQGKRNPKSPLACTNKRPRPEGMQTLESFFKPLTH.

In some cases, a suitable error-prone DNA polymerase comprises an amino acid sequence having at least 85%, at least 90%, at least 95%, at least 98%, at least 99%, or 100%, amino acid sequence identity to a DNA polymerase eta (η) having the following amino acid sequence:

(SEQ ID NO: 77)
MATGQDRVVALVDMDCFFVQVEQRQNPHLRNKPCAVVQYKSWKGGGIIAVSYEARAFGVTRSM
WADDAKKLCPDLLLAQVRESRGKANLTKYREASVEVMEIMSRFAVIERASIDEAYVDLTSAVQER
LQKLQGQPISADLLPSTYIEGLPQGPTTAEETVQKEGMRKQGLFQWLDSLQIDNLTSPDLQLTVG
AVIVEEMRAAIERETGFQCSAGISHNKVLAKLACGLNKPNRQTLVSHGSVPQLFSQMPIRKIRSLG
GKLGASVIEILGIEYMGELTQFTESQLQSHFGEKNGSWLYAMCRGIEHDPVKPRQLPKTIGCSKNF
PGKTALATREQVQWWLLQLAQELEERLTKDRNDNDRVATQLVVSIRVQGDKRLSSLRRCCALTR
YDAHKMSHDAFTVIKNCNTSGIQTEWSPPLTMLFLCATKFSASAPSSSTDITSFLSSDPSSLPKVPV
TSSEAKTQGSGPAVTATKKATTSLESFFQKAAERQKVKEASLSSLTAPTQAPMSN;
and has a length of 511 amino acids.

In some cases, a suitable error-prone DNA polymerase comprises an amino acid sequence having at least 85%, at least 90%, at least 95%, at least 98%, at least 99%, or 100%, amino acid sequence identity to a DNA polymerase kappa (κ) having the following amino acid sequence:

(SEQ ID NO: 13)
MDSTKEKCDSYKDDLLLRMGLNDNKAGMEGLDKEKINKIIMEATKGSRFYGNELKKEKQVNQR
IENMMQQKAQITSQQLRKAQLQVDRFAMELEQSRNLSNTIVHIDMDAFYAAVEMRDNPELKDKP
IAVGSMSMLSTSNYHARRFGVRAAMPGFIAKRLCPQLIIVPPNFDKYRAVSKEVKEILADYDPNF
MAMSLDEAYLNITKHLEERQNWPEDKRRYFIKMGSSVENDNPGKEVNKLSEHERSISPLLFEESP
SDVQPPGDPFQVNFEEQNNPQILQNSVVFGTSAQEVVKEIRFRIEQKTTLTASAGIAPNTMLAKVC
SDKNKPNGQYQILPNRQAVMDFIKDLPIRKVSGIGKVTEKMLKALGIITCTELYQQRALLSLLFSE
TSWHYFLHISLGLGSTHLTRDGERKSMSVERTFSEINKAEEQYSLCQELCSELAQDLQKERLKGR
TVTIKLKNVNFEVKTRASTVSSVVSTAEEIFAIAKELLKTEIDADFPHPLRLRLMGVRISSFPNEED
RKHQQRSIIGFLQAGNQALSATECTLEKTDKDKFVKPLEMSHKKSFFDKKRSERKWSHQDTFKC
EAVNKQSFQTSQPFQVLKKKMNENLEISENSDDCQILTCPVCFRAQGCISLEALNKHVDECLDGP
SISENFKMFSCSHVSATKVNKKENVPASSLCEKQDYEAHPKIKEISSVDCIALVDTIDNSSKAESID
ALSNKHSKEECSSLPSKSFNIEHCHQNSSSTVSLENEDVGSFRQEYRQPYLCEVKTGQALVCPVC
NVEQKTSDLTLFNVHVDVCLNKSFIQELRKDKFNPVNQPKESSRSTGSSSGVQKAVTRTKRPGLM
TKYSTSKKIKPNNPKHTLDIFFK.

In some cases, a suitable error-prone DNA polymerase comprises an amino acid sequence having at least 85%, at least 90%, at least 95%, at least 98%, at least 99%, or 100%, amino acid sequence identity to a DNA polymerase kappa (κ) having the following amino acid sequence:

(SEQ ID NO: 78)
MDSTKEKCDSYKDDLLLRMGLNDNKAGMEGLDKEKINKIIMEATKGSRFYGNELKKEKQVNQR
IENMMQQKAQITSQQLRKAQLQVDRFAMELEQSRNLSNTIVHIDMDAFYAAVEMRDNPELKDKP
IAVGSMSMLSTSNYHARRFGVRAAMPGFIAKRLCPQLIIVPPNFDKYRAVSKEVKEILADYDPNF
MAMSLDEAYLNITKHLEERQNWPEDKRRYFIKMGSSVENDNPGKEVNKLSEHERSISPLLFEESP
SDVQPPGDPFQVNFEEQNNPQILQNSVVFGTSAQEVVKEIRFRIEQKTTLTASAGIAPNTMLAKVC
SDKNKPNGQYQILPNRQAVMDFIKDLPIRKVSGIGKVTEKMLKALGIITCTELYQQRALLSLLFSE
TSWHYFLHISLGLGSTHLTRDGERKSMSVERTFSEINKAEEQYSLCQELCSELAQDLQKERLKGR
TVTIKLKNVNFEVKTRASTVSSVVSTAEEIFAIAKELLKTEIDADFPHPLRLRLMGVRISSFPNEED
RKHQQRSIIGFLQAGNQALSATECTLEKTDKDKFVKPLE; and has a length of 560
amino acids.

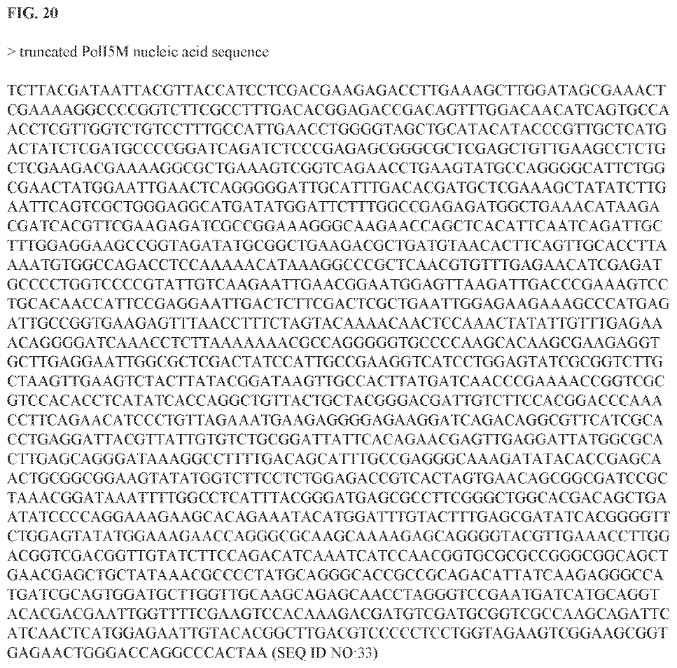

In some cases, a suitable error-prone DNA polymerase comprises an amino acid sequence having at least 85%, at least 90%, at least 95%, at least 98%, at least 99%, or 100%, amino acid sequence identity to the truncated PolI5M nucleic acid sequence depicted in FIG. 20.

### Linkers

In some cases, a fusion polypeptide of the present disclosure comprises a linker positioned between the DNA polymerase and the RNA-guided endonuclease.

In some embodiments, a subject fusion polypeptide can be fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G)ₙ, glycine-serine polymers (including, for example, (GS)ₙ, (GSGGS)ₙ (SEQ ID NO:79), (GGSGGS)ₙ (SEQ ID NO:80), and (GGGGS)ₙ (SEQ ID NO:81), where n is an integer of at least one), glycine-alanine polymers, alanine-serine polymers. Exemplary linkers can comprise amino acid sequences including, but not limited to, GGSG (SEQ ID NO:82), GGSGG (SEQ ID NO:83), GSGSG (SEQ ID NO:84), GSGGG (SEQ ID NO:85), GGGSG (SEQ ID NO:86), GSSSG (SEQ ID NO:87), and the like. The ordinarily skilled artisan will recognize that design of a peptide conjugated to any desired element can include linkers that are all or partially flexible, such that the linker can include a flexible linker as well as one or more portions that confer less flexible structure.

### CRISPR-Cas Guide Nucleic Acids

As noted above, a guide nucleic acid comprises: i) a binding region that binds to a CRISPR-Cas effector polypeptide; and ii) a targeting region that comprises a nucleotide sequence that is complementary to a target sequence of a target nucleic acid. In some cases, the binding region is heterologous to the targeting region. In some cases, the nucleotide sequence that is complementary to a target sequence of a target nucleic acid is 15 nucleotides to 19 nucleotides long. In some cases, the nucleotide sequence that is complementary to a target sequence of a target nucleic acid is 18 nucleotides long. The nucleotide sequence that is complementary to a target sequence of a target nucleic acid is no more than 19 nucleotides long, e.g., no more than 18 nucleotides long.

A guide RNA that includes a nucleotide sequence that is complementary to a target sequence of a target nucleic acid, where the nucleotide sequence is no more than 18 nucleotides long, provides for an increased mutation rate. For example, a guide RNA that includes an 18-nucleotide sequence that is complementary to a target sequence of a target nucleic acid, when complexed with a fusion polypeptide of the present disclosure (where the fusion polypeptide comprises an error-prone DNA polymerase and a CRISPR-Cas nickase polypeptide), provides for a mutation rate that is from 2-fold to 1000-fold higher (e.g., from 2-fold to 5-fold, from 5-fold to 10-fold, from 10-fold to 25-fold, from 25-fold to 50-fold, from 50-fold to 100-fold, from 100-fold to 250-fold, from 250-fold to 500-fold, or from 500-fold to 1000-fold higher) than the mutation rate obtained when the guide RNA includes a 20-nucleotide sequence that is complementary to the target sequence of the target nucleic acid.

In some cases, a CRISPR-Cas effector guide RNA has one or more modifications, e.g., one or more of a base modification, a backbone modification, and a sugar modification.

Suitable modified backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′,5′ to 5′ or 2′ to 2′ linkage. Suitable nucleic acids having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂ CH₂OCH₃, also known as 2′-O-(2-methoxyethyl) or 2′-MOE) (Martin et al., Helv. Chim. Acta, 1995, 78, 486-504, the disclosure of which is incorporated herein by reference in its entirety) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

### Systems

The present disclosure provides systems for carrying out a method of the present disclosure.

In some cases, a system of the present disclosure comprises: a) a fusion polypeptide comprising: i) a CRISPR-Cas effector polypeptide that exhibits nickase activity; and ii) two or more heterologous polypeptides, wherein one of the two or more heterologous polypeptides is an error-prone DNA polymerase that lacks all or a portion of a 3′-to-5′ exonuclease domain and/or lacks all or a portion of a 5′-to-3′ exonuclease domain, wherein one of the two or more heterologous polypeptides comprises an NES polypeptide, and wherein the two or more heterologous polypeptides does not include a nuclear localization signal (NLS) polypeptide; and b) one or more guide nucleic acids, wherein one or more guide nucleic acids comprise: i) a targeting region that comprises a nucleotide sequence that binds to a target sequence in the target viral nucleic acid, wherein the nucleotide sequence that binds to the target sequence has a length of from 15 nucleotides to 18 nucleotides; and ii) a protein-binding region that binds to the CRISPR-Cas effector polypeptide. In some cases, an error-prone DNA polymerase lacking all or a portion of a 3′-to-5′ exonuclease domain comprises an amino acid sequence having at least 85%, at least 90%, at least 95%, at least 98%, at least 99%, or 100%, amino acid sequence identity to the amino acid sequence depicted in FIG. 1F.

In some cases, a system of the present disclosure comprises: a) a nucleic acid (e.g., a recombinant expression vector) comprising a nucleotide sequence encoding a fusion polypeptide comprising: i) a CRISPR-Cas effector polypeptide that exhibits nickase activity; and ii) two or more heterologous polypeptides, wherein one of the two or more heterologous polypeptides is an error-prone DNA polymerase that lacks all or a portion of a 3′-to-5′ exonuclease domain and/or lacks all or a portion of a 5′-to-3′ exonuclease domain, wherein one of the two or more heterologous polypeptides comprises an NES polypeptide, and wherein the two or more heterologous polypeptides does not include an NLS polypeptide; and b) one or more guide nucleic acids, wherein one or more guide nucleic acids comprise: i) a targeting region that comprises a nucleotide sequence that binds to a target sequence in the target viral nucleic acid, wherein the nucleotide sequence that binds to the target sequence has a length of from 15 nucleotides to 18 nucleotides; and ii) a protein-binding region that binds to the CRISPR-Cas effector polypeptide.

In some cases, a system of the present disclosure comprises: a) a nucleic acid (e.g., a recombinant expression vector) comprising: 1) a first nucleotide sequence encoding a fusion polypeptide comprising: i) a CRISPR-Cas effector polypeptide that exhibits nickase activity; and ii) two or more heterologous polypeptides, wherein one of the two or more heterologous polypeptides is an error-prone DNA polymerase that lacks all or a portion of a 3′-to-5′ exonuclease domain and/or lacks all or a portion of a 5′-to-3′ exonuclease domain, wherein one of the two or more heterologous polypeptides comprises an NES polypeptide, and wherein the two or more heterologous polypeptides does not include an NLS polypeptide; and 2) a second nucleotide sequence encoding one or more guide nucleic acids, wherein one or more guide nucleic acids comprise: i) a targeting region that comprises a nucleotide sequence that binds to a target sequence in the target viral nucleic acid, wherein the nucleotide sequence that binds to the target sequence has a length of from 15 nucleotides to 18 nucleotides; and ii) a protein-binding region that binds to the CRISPR-Cas effector polypeptide.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method of modifying a target viral nucleic acid in the cytoplasm of a eukaryotic cell, the method comprising contacting the target viral nucleic acid with: a) a fusion polypeptide comprising: i) a CRISPR-Cas effector polypeptide; and ii) two or more heterologous polypeptides, wherein one of the two or more heterologous polypeptides is an error-prone DNA polymerase and wherein one of the two or more heterologous polypeptides comprises a nuclear export signal (NES) polypeptide; and b) one or more guide nucleic acids, wherein one or more guide nucleic acids comprise: i) a targeting region that comprises a nucleotide sequence that binds to a target sequence in the target viral nucleic acid; and ii) a protein-binding region that binds to the CRISPR-Cas effector polypeptide, wherein said contacting provides for modification of the target viral nucleic acid.

Aspect 2. The method of aspect 1, wherein the fusion polypeptide does not include a nuclear localization signal (NLS).

Aspect 3. The method of aspect 1 or aspect 2, wherein the targeting region of the guide nucleic acid has a length of from about 15 nucleobases to 19 nucleobases.

Aspect 4. The method of any one of aspects 1-3, wherein the error-prone DNA polymerase lacks 3′-5′ exonuclease activity and/or lacks 5′-to-3′ exonuclease activity.

Aspect 5. The method of any one of aspects 1-4, wherein the fusion polypeptide has a length of no more than about 3000 amino acids.

Aspect 6. The method of any one of aspects 1-5, further comprising contacting the target viral nucleic acid with a donor nucleic acid.

Aspect 7. The method of any one of aspects 1-6, wherein the CRISPR-Cas effector polypeptide is a type II CRISPR-Cas effector polypeptide, a type III CRISPR-Cas effector polypeptide, a type IV CRISPR-Cas effector polypeptide, a type V CRISPR-Cas effector polypeptide, or a type VI CRISPR-Cas effector polypeptide.

Aspect 8. The method of any one of aspects 1-6, wherein the CRISPR-Cas effector polypeptide is a Cas9 polypeptide.

Aspect 9. The method of aspect 8, wherein the Cas9 polypeptide is a variant Cas9 polypeptide that has relaxed protospacer adjacent motif (PAM) requirements, optionally wherein the variant Cas9 polypeptide comprises an amino acid sequence having at least 50% amino acid sequence identity to the amino acid sequence depicted in any one of FIG. 14A-14D.

Aspect 10. The method of any one of aspects 1-9, wherein the fusion polypeptide, when complexed with a guide RNA, exhibits a target mutation rate of from 10⁻⁸ to 10⁻² mutations per nucleotide per viral genome replication event.

Aspect 11. The method of any one of aspects 1-9, wherein the fusion polypeptide, when complexed with a guide RNA, exhibits a target mutation rate of from 10⁻⁶ to 10⁻⁵ mutations per nucleotide per viral genome replication event.

Aspect 12. The method of any one of aspects 1-9, wherein the fusion polypeptide, when complexed with a guide RNA, exhibits a target mutation rate of from 10⁻⁵ to 10⁻³ mutations per nucleotide per viral genome replication event.

Aspect 13. The method of any one of aspects 1-9, wherein the fusion polypeptide, when complexed with a guide RNA, exhibits a target mutation rate of from 10⁻³ to 10⁻² mutations per nucleotide per viral genome replication event.

Aspect 14. The method of any one of aspects 1-13, wherein the CRISPR-Cas effector polypeptide is a nickase.

Aspect 15. The method of any one of aspects 1-13, wherein the CRISPR-Cas effector polypeptide lacks catalytic activity but retains binding to the target viral nucleic acid.

Aspect 16. The method of any one of aspects 1-15, wherein the target viral nucleic acid is a nucleic acid of a double-stranded DNA virus that has a genome length of from about 50 kbp to about 1.2 mbp, or from about 150 kbp to 1.2 mbp, and wherein at least part of the replication cycle of the double-stranded DNA virus occurs in the cytoplasm of the cell.

Aspect 17. The method of aspect 16, wherein the double-stranded DNA virus is a virus of a family selected from Poxviridae, Asfaviridae, Iridoviridae, Ascovirida, Phycodnaviridae, Marseilleviridae, Pithoviridae, Mimiviridae, Pandoraviridae, Molliviruses, and Faustoviruses.

Aspect 18. The method of any one of aspects 1-17, wherein the DNA polymerase comprises an amino acid sequence having at least 85% amino acid sequence to the DNA polymerase I amino acid sequence depicted in FIG. 1A or FIG. 1F, wherein the DNA polymerase has one or more of the following: an Ala at amino acid position 424, an Asn at amino acid position 709, a Tyr at amino acid position 742, an Arg at amino acid position 759, and a His at amino acid position 796, optionally wherein the DNA polymerase lacks all or a portion of a 3′-to-5′ exonuclease domain and/or lacks all or a portion of a 5′-to-3′ exonuclease domain.

Aspect 19. The method of any one of aspects 1-17, wherein the DNA polymerase is a DNA polymerase beta, a DNA polymerase iota, a DNA polymerase nu, a DNA polymerase eta, or a DNA polymerase kappa.

Aspect 20. The method of any one of aspects 1-19, wherein the fusion polypeptide, when complexed with a guide RNA, exhibits a target mutation rate of 1 mutation per nucleotide per viral genome replication event.

Aspect 21. The method of any one of aspects 1-20, wherein the method comprises introducing into the eukaryotic cell a recombinant expression construct that comprises a nucleotide sequence encoding the fusion polypeptide.

Aspect 22. The method of aspect 21, wherein the recombinant expression construct comprises a nucleotide sequence encoding the guide RNA.

Aspect 23. A method of modifying a target viral nucleic acid in the cytoplasm of a eukaryotic cell, the method comprising: A) introducing into the eukaryotic cell gene editing components, wherein the gene editing components comprise: a) a fusion polypeptide comprising: i) a CRISPR-Cas effector polypeptide; and ii) two or more heterologous polypeptides, wherein one of the two or more heterologous polypeptides is an error-prone DNA polymerase and wherein one of the two or more heterologous polypeptides comprises a nuclear export signal (NES) polypeptide; and b) one or more guide nucleic acids, wherein one or more guide nucleic acids comprise: i) a targeting region that comprises a nucleotide sequence that binds to a target sequence in the target viral nucleic acid; and ii) a protein-binding region that binds to the CRISPR-Cas effector polypeptide, thereby generating a modified eukaryotic cell; and B) infecting the modified eukaryotic cell with a virus comprising the target viral nucleic acid, wherein the target viral nucleic acid is contacted with the gene editing components, and wherein said contacting provides for modification of the target viral nucleic acid.

Aspect 24. A system for modifying a target viral nucleic acid in the cytoplasm of a eukaryotic cell, the system comprising:
a1) a fusion polypeptide comprising: i) a CRISPR-Cas effector polypeptide that exhibits nickase activity; and ii) two or more heterologous polypeptides, wherein one of the two or more heterologous polypeptides is an error-prone DNA polymerase that lacks all or a portion of a 3′-to-5′ exonuclease domain and/or lacks all or a portion of a 5′-to-3′ exonuclease domain, wherein one of the two or more heterologous polypeptides comprises a nuclear export signal (NES) polypeptide, and wherein the two or more heterologous polypeptides does not include a nuclear localization signal (NLS) polypeptide; and
b1) one or more guide nucleic acids, wherein one or more guide nucleic acids comprise: i) a targeting region that comprises a nucleotide sequence that binds to a target sequence in the target viral nucleic acid, wherein the nucleotide sequence that binds to the target sequence has a length of from 15 nucleotides to 18 nucleotides; and ii) a protein-binding region that binds to the CRISPR-Cas effector polypeptide; or
a2) a nucleic acid comprising a nucleotide sequence encoding a fusion polypeptide comprising: i) a CRISPR-Cas effector polypeptide that exhibits nickase activity; and ii) two or more heterologous polypeptides, wherein one of the two or more heterologous polypeptides is an error-prone DNA polymerase that lacks all or a portion of a 3′-to-5′ exonuclease domain and/or lacks all or a portion of a 5′-to-3′ exonuclease domain, wherein one of the two or more heterologous polypeptides comprises an NES polypeptide, and wherein the two or more heterologous polypeptides does not include an NLS polypeptide; and
b2) one or more guide nucleic acids, wherein one or more guide nucleic acids comprise: i) a targeting region that comprises a nucleotide sequence that binds to a target sequence in the target viral nucleic acid, wherein the nucleotide sequence that binds to the target sequence has a length of from 15 nucleotides to 19 nucleotides; and ii) a protein-binding region that binds to the CRISPR-Cas effector polypeptide; or
a3) a nucleic acid comprising:
1) a first nucleotide sequence encoding a fusion polypeptide comprising: i) a CRISPR-Cas effector polypeptide that exhibits nickase activity; and ii) two or more heterologous polypeptides, wherein one of the two or more heterologous polypeptides is an error-prone DNA polymerase that lacks all or a portion of a 3′-to-5′ exonuclease domain and/or lacks all or a portion of a 5′-to-3′ exonuclease domain, wherein one of the two or more heterologous polypeptides comprises an NES polypeptide, and wherein the two or more heterologous polypeptides does not include an NLS polypeptide; and
2) a second nucleotide sequence encoding one or more guide nucleic acids, wherein one or more guide nucleic acids comprise: i) a targeting region that comprises a nucleotide sequence that binds to a target sequence in the target viral nucleic acid, wherein the nucleotide sequence that binds to the target sequence has a length of from 15 nucleotides to 18 nucleotides; and ii) a protein-binding region that binds to the CRISPR-Cas effector polypeptide.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

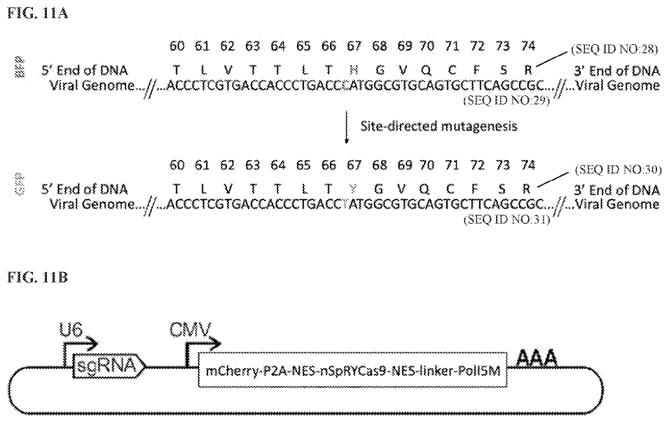

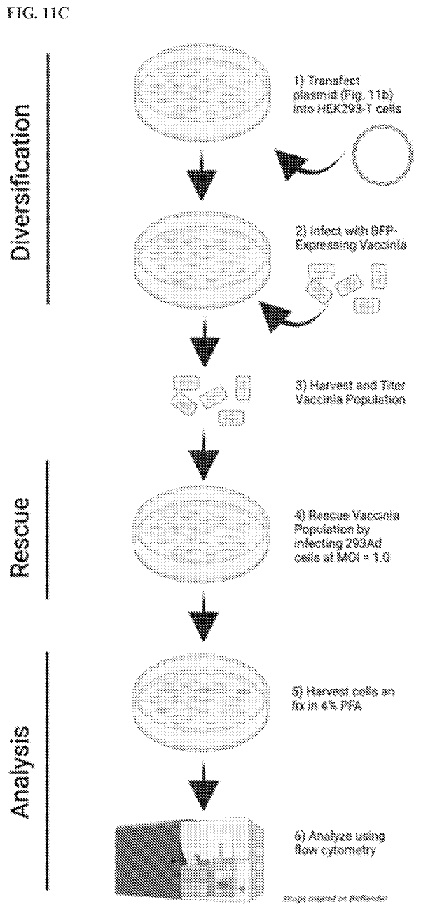

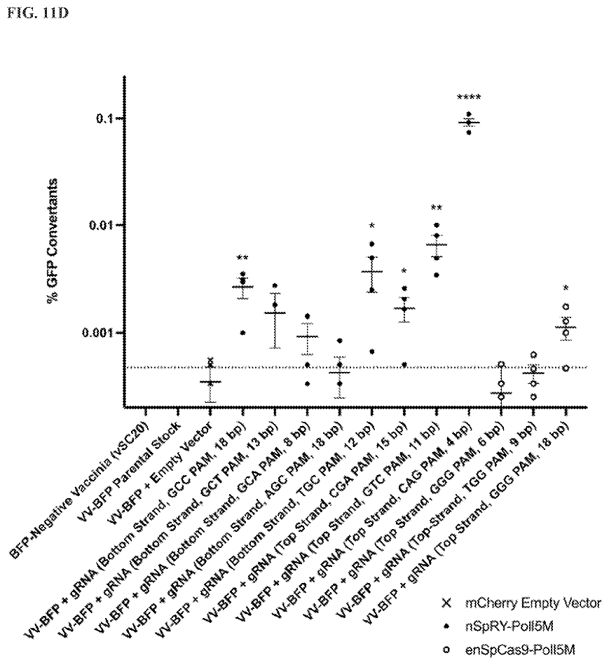

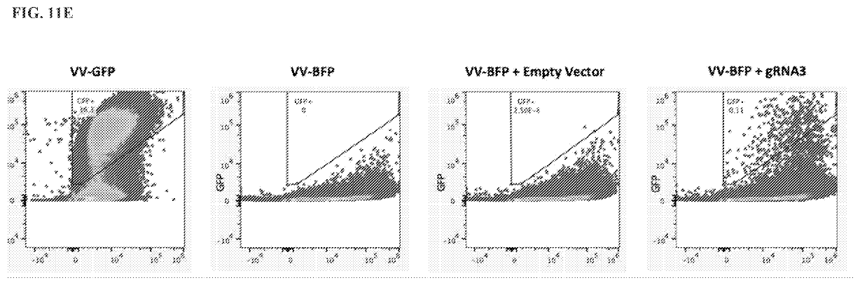

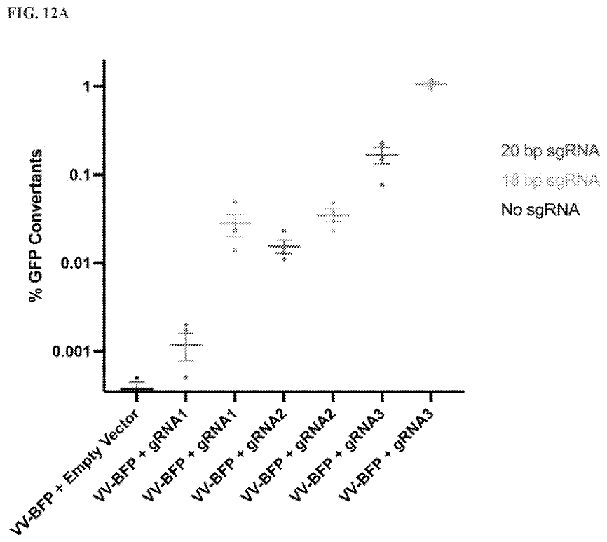

As an initial proof-of-concept to illustrate the idea and estimate the mutation rate of generating random mutagenesis within a user-defined locus in the genome of an NCLDV, a blue fluorescent protein (BFP) to green fluorescent protein (GFP) conversion assay (Glaser et al. (2016) Molecular Therapy—Nucleic Acids 5:e334) was adapted; this assay enables the visualization and quantification of a Histidine to Tyrosine (H68Y) amino acid substitution that results from a single Cytosine to Thymine nucleotide substitution (FIG. 11A). A bfp gene sequence was incorporated into the J2R locus of vaccinia virus strain western reserve for which the gene products J2R (encoding thymidine kinase (TK), which is critical for nucleotide precursor processing) and both copies of C11R (encoding vaccinia growth factor (VGF), which promotes bystander cells to proceed into S phase) were knocked out to limit replication in non-cancerous cells and thus ensure safety (VV-BFP). A plasmid driving expression of sgRNAs targeted to the bfp region ofVV-BFP, and a plasmid encoding an mCherry-tagged fusion protein consisting of an RNA-guided nickase fused to an error-prone DNA polymerase, were generated.

Three engineering improvements were essential to achieve useful activity levels of such a fusion protein in cytoplasmic DNA viral genomes, including poxviruses and vaccinia: 1. A D10A mutation was made in the SpRY Cas9 variant such that the A-T rich genome of poxviruses could be targeted in all loci, 2. A nuclear export sequence (NES) was used in place of a nuclear localization sequence (NLS), to localize the complex to the cytoplasmic site of NCLDV genome replication, and 3. The template binding regions of each sgRNA was truncated from the canonical 20 bp down to 18 bp, which significantly increased on-target observed mutation rate.

To test the highly engineered complexes, each plasmid was transfected into human embryonic kidney (HEK293-T) cells. Next, cells expressing the editing system were infected with VV-BFP at 24 hours post-transfection (HPT). Crude lysate was subsequently harvested, freeze-thawed three times, and rescued by infecting HEK293-Ad cells with the crude lysate at MOI=0.1. Following rescue, cells were fixed with 4% PFA, GFP reversion rates were quantified by flow cytometry using an Attune flow cytometer, and the data were analyzed using FlowJo software (FIG. 11B). Initial results showed nuclease- and sgRNA-dependent GFP reversion efficiencies ranging from near the background reversion rate of virus passaged without the editing system up to three orders of magnitude higher (0.108%) than background (FIG. 11C). The use of enhanced nicking S. pyogenes Cas9 (rather than nicking SpRY Cas9) fused to PolI5M showed low GFP reversion rates. Upon optimizing the system by using a nickase with flexible PAM usage, tagging the molecule with two strategically placed NES sequences, and truncating the template binding regions of sgRNAs from 20 bp to 18 bp, all tested sgRNAs conferred over 0.1% GFP reversion rates, with the highest performing sgRNA conferring 1.18% GFP reversion after a single passage of virus on HEK293-T cells expressing the editing system (FIG. 11D).

FIG. 11A-11E. System for in vivo diversification of user-defined loci in cytoplasmic DNA using the poxvirus vaccinia as a model. A) A measurable shift in emission can be observed through the generation of an H67Y (BFP->GFP) mutation, which is rendered by a single C->T nucleic acid substitution. B) Map showing plasmid expression system of the RNA-guided fusion protein used for site-specific diversification of NCLDV genomes in mammalian cells. C) Methods for proof-of-concept experiment assessing the diversification of a bfp gene that has been incorporated into the J2R locus of the vaccinia genome (VV-BFP). GFP reversion rates represent just one of many possible nucleotide substitutions and therefore provide an estimate of diversification efficiency. D) GFP reversion rates assessed using twelve different sgRNAs and two different nickases show both utility and non-obviousness of the invention. Each sample represents four independently transfected, infected, and rescued biological replicates. Statistical significance of individual samples relative to the empty vector control were calculated by a student's t-test. E) Representative dot plots for positive gating control (VV-GFP=vaccinia stably expressing the corresponding gfp from the J2R locus) and two of the negative controls (VV-BFP virus that was not passaged on cells and VV-BFP that was passaged on cells expressing an mCherry-only empty vector).

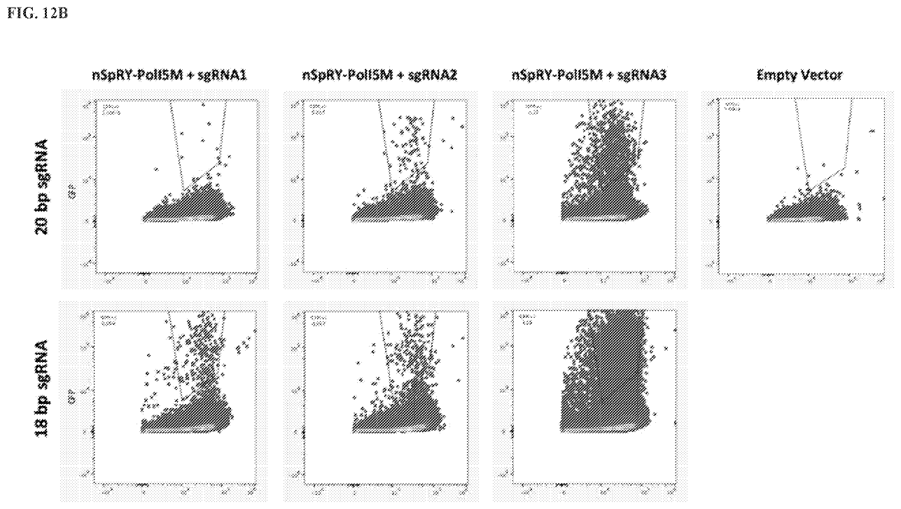

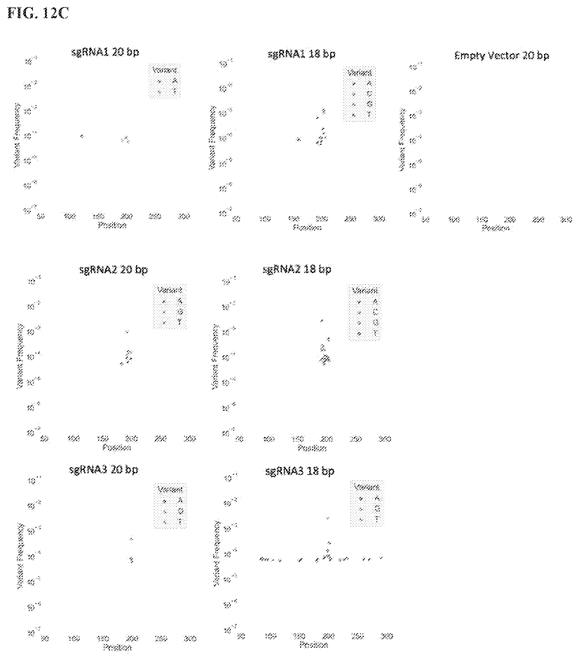

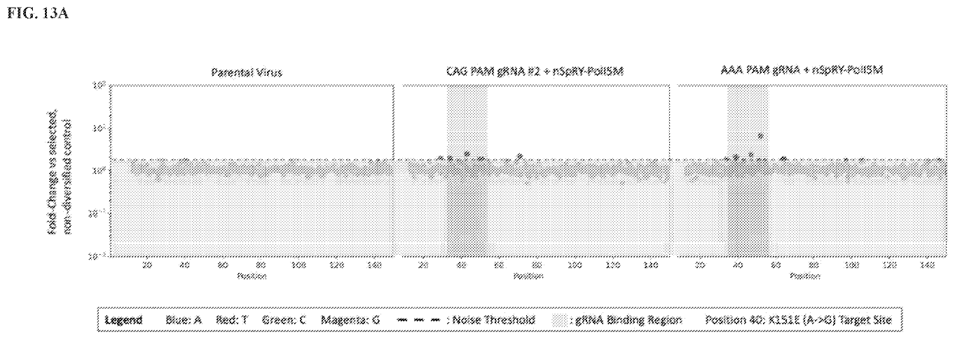

FIG. 12A-12C. Truncation of PAM-distal base pairs from sgRNA template binding region increases nSpRY-PolI5M-mediated effective SNP generation. A) Head-to-head quantification of GFP reversion rates in a VV-BFP population. Four independently transfected/infected biological replicates show that a two base pair truncation from the PAM-distal side of the sgRNA binding region increase GFP reversion rates for all sgRNAs, regardless of their initial performance. B) Representative dot plots of an individual biological replicate shown in FIG. 12A. C) Genomic DNAs of VV-BFP populations depicted in FIG. 12A were harvested, amplified by PCR, and submitted for Illumina sequencing. Rare variant analysis for which the variant frequency of the empty vector condition was subtracted from each condition showed an increase in observed mutations in viral populations passaged on the truncated (18 bp template binding region-containing) sgRNA samples relative to the full-length (20 bp template binding region-containing) sgRNA conditions.

To further illustrate proof-of-concept of the editing system, we designed a second functional assay involving the conserved vaccinia gene A34R. A34R encodes a type II integral membrane glycoprotein with critical functions in the cellular release and infectivity of EEV. Several prior studies offer proof-of-principle that point mutations in A34R can improve oncolytic vaccinia by increasing EEV production. First, the IHD-J strain of vaccinia naturally produces up to 30% of virions in the EEV form16 due to differences in only two residues—in stark contrast to the <1% produced by other vaccinia strains, including WR. When tested in an oncolytic context, vaccinia WR expressing the IHD-J A34 protein variant in place of its endogenous A34 showed enhanced spread from subcutaneous tumors to lung tumors in vivo. Moreover, a single, rationally designed K151E point mutation to A34R of WR vaccinia was shown to increase EEV production and resulted in improved spread and replication of an oncolytic vaccinia backbone in a peritoneal carcinomatosis model of MC38 colon cancer. Two sgRNAs were designed with 20 bp template binding regions targeted to the locus that encodes for Lysine at the 151st amino acid position ofA34R and subjected the sequences to NGS. Initial results show that the non-optimized version of the editing system created observable diversity within the targeting window. Next, the optimized, truncated sgRNAs were used to guide nSpRY-PolI5M to the locus. Three iterative rounds of diversification were performed, followed by selection with the IMV-neutralizing antibody 7D11. NGS and functional analysis of the results of this experiment are currently in progress. Collectively, these data represent the first evidence of user-defined, targeted diversification of cytoplasmic DNA in mammalian cells.

Some NCLDV species harbor AT-rich genomes. To enable targeted diversification of any locus, the capability of the technology to target an AT-rich region of the A34R gene of vaccinia virus (containing C11R and J2R gene deletions) was tested.

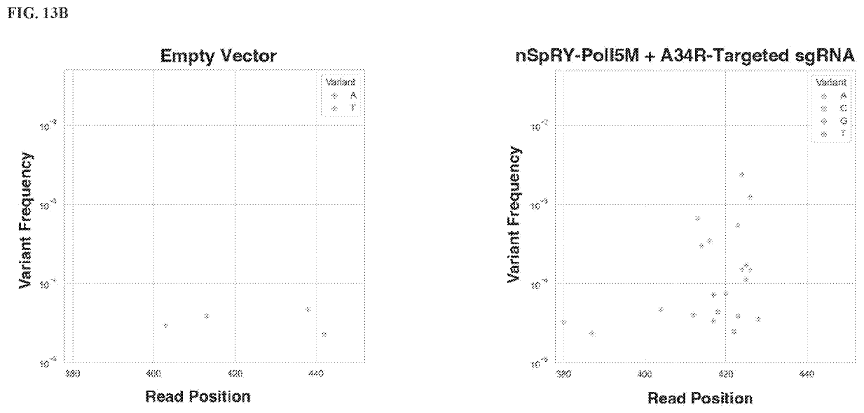

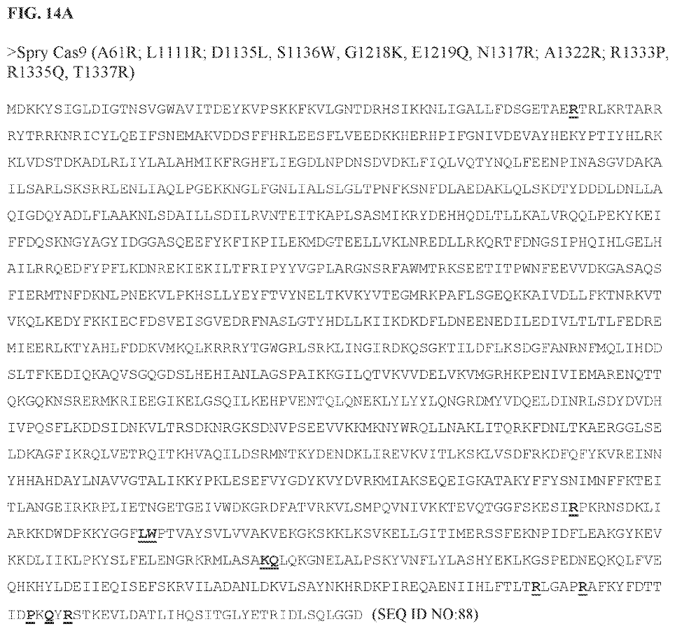

FIG. 13A-13B. nSpRY-PolI5M fusion complex guided by full-length (non-truncated) sgRNAs or truncated (18 bp) sgRNAs conferred on-target mutagenesis of an AT-rich endogenous locus in the vaccinia virus genome. A) A nSpRY-PolI5M guided by full-length (20 bp target site-binding) sgRNAs creates site-specific diversity detectable with 100,000 reads. B) To enable targeted diversification of any locus, the capability of the technology to target an AT-rich region of the A34R gene of vaccinia virus when using truncated (18 bp target site-binding) gRNAs was tested. Two passages of VV-GFP on HEK293-T cells transiently transfected with nSpRY-PolI5M and an on-target sgRNA exhibited elevated diversity (relative to VV-GFP passaged on HEK293-T transfected with empty vector) in the endogenous A34R locus when analyzed by Illumina amplicon sequencing. Collectively, these data provide further evidence demonstrating targeted diversification of user-defined loci within cytoplasmic DNA in mammalian cells.

### Example 2

Head-to-head comparisons of GFP conversion rates (quantified by flow cytometry) were performed to assess the off-target effects of the CRISPR fusion protein editing complex. VV-BFP passaged a single time on HEK293T cells expressing nSpRY and sgRNA targeted to the codon encoding HIS67 of BFP but lacking a fused polymerase did not contain elevated mutations relative to the unpassaged VV-BFP or VV-BFP passaged on cells transfected with an empty vector.

FIG. 15 RNA-guided nSpRY-PolI5M fusion complex conferred on-target mutagenesis of VV-BFP with low off-target effects. Conditions that contained nSpRY-PolI5M fusion protein and a sgRNA targeted to off-target or non-target sites conferred slight elevation in GFP conversion relative to background, which was not dependent upon the distance of the sgRNA target site from the site encoding the HIS67 of BFP. On the other hand, only conditions that contained the nSpRY-PolI5M fusion protein with the on-target sgRNA alone or on-target sgRNA co-transfected with a pool of three off-target gRNAs conferred GFP conversion rates over three orders of magnitude higher than that of VV-BFP's background rate of GFP conversion. All conditions depicted in FIG. 15 utilized n=4 independent biological replicates, and all sgRNAs utilized 18 bp template-binding regions. Statistical significance of each indicated group was calculated by one-way ANOVA. These data show the necessity of both an on-target gRNA and nickase-polymerase fusion complex to achieve elevated diversification at a targeted locus in a model NCLDV.

To show the technology works broadly among NCLDVs, the ability of nSpRY-PolI5M to target a gene encoding GFP that was stably incorporated into the genome of a distantly related poxvirus known as myxoma virus (MYXV) was tested. Myxoma virus is a Leporipoxvirus, a genus of poxviruses whose host range is restricted to Lagomorphs and squirrels. On the other hand, vaccinia is an Orthopoxvirus and contains numerous gene deletions, additions, and mutations that account for its expanded host range. Both viruses have been explored as anti-cancer agents, and MYXV was historically used as a biological control agent against invasive European Rabbits on the Australian continent.

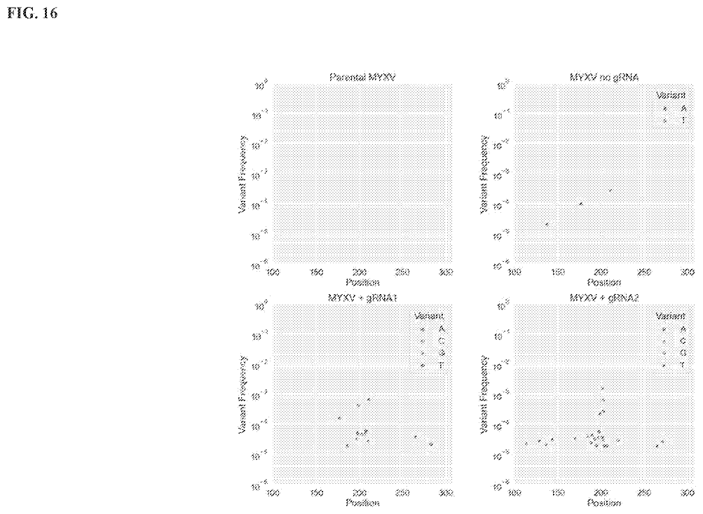

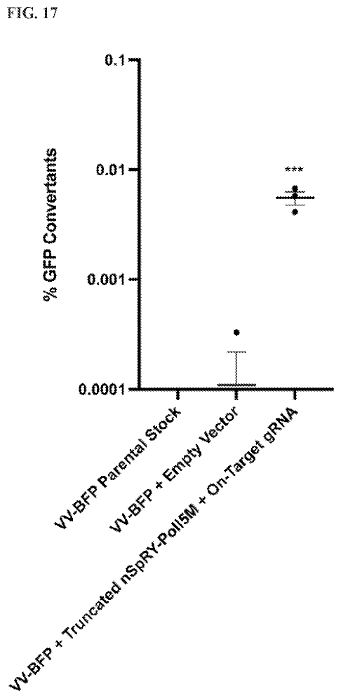

FIG. 16 RNA-guided nSpRY-PolI5M fusion complex conferred on-target mutagenesis in the genome of a distantly related poxvirus species, myxoma virus. A single passage of MYXV on HEK293 cells transiently transfected with nSpRY-PolI5M and a sgRNA targeted to the incorporated gene encoding GFP exhibited elevated diversity at the target locus when analyzed by Illumina amplicon sequencing. These data show the technology works in a broad range of NCLDVs.

A potential use of this technology is to generate targeted, continuous diversity of cytoplasmic DNA in vivo. However, leading gene delivery systems, such as adenovirus or lentivirus, needed to enable stable gene expression of the RNA-guided fusion protein in vivo, suffer from limited coding capacity for heterologous genetic cargo. Thus, whether truncated versions of the nickase and polymerase could diversify the codon encoding HIS67 of BFP in VV-BFP were tested.

FIG. 17 Truncated nuclease and polymerase complex generated elevated diversity at a targeted locus in VV-BFP. A single passage of MYXV-GFP on HEK293 cells transiently transfected with a plasmid encoding an on-target sgRNA and a fusion protein comprised of a truncated nSpRY and truncated PolISM exhibited elevated GFP conversion when analyzed by flow cytometry. These data show a truncated version of the technology that is within the packaging limit of standard lentivirus vectors retains functional activity.

To assess multiplexed on-target mutagenesis, VV-BFP, for which the gene products J2R (encoding thymidine kinase (TK), which is critical for nucleotide precursor processing) and both copies of C₁₁R (encoding vaccinia growth factor (VGF), which promotes bystander cells to proceed into S phase) were knocked out to limit replication in non-cancerous cells and thus ensure safety, was passaged on HEK293-T cells expressing nSpRY-PolI5M and a pool of 39 sgRNAs whose target sites were tiled throughout the A34R gene. Following two rounds of passaging on the cells expressing the editing machinery, viral genomic DNA was harvested, and a −200 bp window of the A34R locus was PCR-amplified and analyzed by next-generation sequencing.

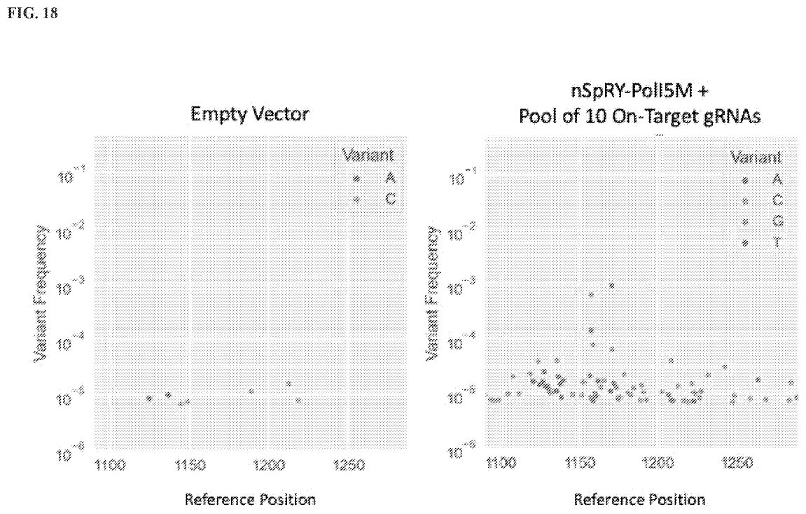

FIG. 18 The nSpRY-PolI5M fusion protein guided by a pool of 39 sgRNAs generates elevated diversity across an endogenous gene of interest. VV-BFP was passaged HEK293-T cells for which a pool of 39 sgRNAs had been tiled across the A34R locus. Rare variant analysis results show an elevated number of unique mutations in a selected region of the A34R locus for VV-BFP passaged on HEK293-T cells expressing nSpRY-PolI5M and the pool of sgRNAs relative to VV-BFP passaged on HEK293-T cells expressing empty vector. These data show that the technology is multiplexable across an endogenous locus of a model NCLDV genome.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of modifying a target viral nucleic acid in the cytoplasm of a eukaryotic cell, the method comprising contacting the target viral nucleic acid with:
a) a fusion polypeptide comprising: i) a CRISPR-Cas effector polypeptide; and ii) two or more heterologous polypeptides, wherein one of the two or more heterologous polypeptides is an error-prone DNA polymerase and wherein one of the two or more heterologous polypeptides comprises a nuclear export signal (NES) polypeptide; and
b) one or more guide nucleic acids, wherein one or more guide nucleic acids comprise: i) a targeting region that comprises a nucleotide sequence that binds to a target sequence in the target viral nucleic acid; and ii) a protein-binding region that binds to the CRISPR-Cas effector polypeptide,
wherein said contacting provides for modification of the target viral nucleic acid.

2. The method of claim 1, wherein the fusion polypeptide does not include a nuclear localization signal (NLS).

3. The method of claim 1, wherein the targeting region of the guide nucleic acid has a length of from about 15 nucleobases to 19 nucleobases.

4. The method of claim 1, wherein the error-prone DNA polymerase has reduced or lacks 3′-5′ exonuclease activity.

5. The method of claim 1, wherein the fusion polypeptide has a length of no more than about 3000 amino acids.

6. The method of claim 1, further comprising contacting the target viral nucleic acid with a donor nucleic acid.

7. The method of claim 1, wherein the CRISPR-Cas effector polypeptide is a type II CRISPR-Cas effector polypeptide, a type III CRISPR-Cas effector polypeptide, a type IV CRISPR-Cas effector polypeptide, a type V CRISPR-Cas effector polypeptide, or a type VI CRISPR-Cas effector polypeptide.

8. (canceled)

9. The method of claim 1, wherein the CRISPR-Cas effector polypeptide is a Cas9 polypeptide or wherein the CRISPR-Cas effector polypeptide is a variant Cas9 polypeptide that has relaxed protospacer adjacent motif (PAM) requirements, optionally wherein the variant Cas9 polypeptide comprises an amino acid sequence having at least 50% amino acid sequence identity to the amino acid sequence depicted in any one of SEQ ID NOs: 88-90 and 32.

10. The method of claim 1, wherein the fusion polypeptide, when complexed with a guide RNA, exhibits a target mutation rate of from 10⁻⁸ to 10⁻² mutations per nucleotide per viral genome replication event, from 10⁻⁶ to 10⁻⁵ mutations per nucleotide per viral genome replication event, from 10⁻⁵ to 10⁻³ mutations per nucleotide per viral genome replication event, or from 10⁻³ to 10⁻² mutations per nucleotide per viral genome replication event.

11.-13. (canceled)

14. The method of claim 1, wherein the CRISPR-Cas effector polypeptide is a nickase.

15. The method of claim 1, wherein the CRISPR-Cas effector polypeptide lacks catalytic activity but retains binding to the target viral nucleic acid.

16. The method of claim 1, wherein the target viral nucleic acid is a nucleic acid of a double-stranded DNA virus that has a genome length of from about 50 kbp to about 1.2 mbp, or from about 150 kbp to 1.2 mbp, and wherein at least part of the replication cycle of the double-stranded DNA virus occurs in the cytoplasm of the cell.

17. The method of claim 16, wherein the double-stranded DNA virus is a virus of a family selected from Poxviridae, Asfaviridae, Iridoviridae, Ascovirida, Phycodnaviridae, Marseilleviridae, Pithoviridae, Mimiviridae, Pandoraviridae, Molliviruses, and Faustoviruses.

18. The method of claim 1, wherein the DNA polymerase comprises an amino acid sequence having at least 85% amino acid sequence to the DNA polymerase I amino acid sequence depicted in SEQ ID NO:1, wherein the DNA polymerase has one or more of the following: an Ala at amino acid position 424, an Asn at amino acid position 709, a Tyr at amino acid position 742, an Arg at amino acid position 759, and a His at amino acid position 796 or wherein the DNA polymerase is a DNA polymerase beta, a DNA polymerase iota, a DNA polymerase nu, a DNA polymerase eta, or a DNA polymerase kappa.

19. (canceled)

20. The method of claim 1, wherein the fusion polypeptide, when complexed with a guide RNA, exhibits a target mutation rate of 1 mutation per nucleotide per viral genome replication event.

21. The method of claim 1, wherein the method comprises introducing into the eukaryotic cell a recombinant expression construct that comprises a nucleotide sequence encoding the fusion polypeptide.

22. The method of claim 21, wherein the recombinant expression construct comprises a nucleotide sequence encoding the guide RNA.

23. A method of modifying a target viral nucleic acid in the cytoplasm of a eukaryotic cell, the method comprising:
A) introducing into the eukaryotic cell gene editing components, wherein the gene editing components comprise:
a) a fusion polypeptide comprising: i) a CRISPR-Cas effector polypeptide; and ii) two or more heterologous polypeptides, wherein one of the two or more heterologous polypeptides is an error-prone DNA polymerase and wherein one of the two or more heterologous polypeptides comprises a nuclear export signal (NES) polypeptide; and
b) one or more guide nucleic acids, wherein one or more guide nucleic acids comprise: i) a targeting region that comprises a nucleotide sequence that binds to a target sequence in the target viral nucleic acid; and ii) a protein-binding region that binds to the CRISPR-Cas effector polypeptide, thereby generating a modified eukaryotic cell; and
B) infecting the modified eukaryotic cell with a virus comprising the target viral nucleic acid, wherein the target viral nucleic acid is contacted with the gene editing components, and wherein said contacting provides for modification of the target viral nucleic acid.

24. A system for modifying a target viral nucleic acid in the cytoplasm of a eukaryotic cell, the system comprising:
a1) a fusion polypeptide comprising: i) a CRISPR-Cas effector polypeptide that exhibits nickase activity; and ii) two or more heterologous polypeptides, wherein one of the two or more heterologous polypeptides is an error-prone DNA polymerase that lacks all or a portion of a 3′-to-5′ exonuclease domain and/or lacks all or a portion of a 5′-to-3′ exonuclease domain, wherein one of the two or more heterologous polypeptides comprises a nuclear export signal (NES) polypeptide, and wherein the two or more heterologous polypeptides does not include a nuclear localization signal (NLS) polypeptide; and
b1) one or more guide nucleic acids, wherein one or more guide nucleic acids comprise: i) a targeting region that comprises a nucleotide sequence that binds to a target sequence in the target viral nucleic acid, wherein the nucleotide sequence that binds to the target sequence has a length of from 15 nucleotides to 18 nucleotides; and ii) a protein-binding region that binds to the CRISPR-Cas effector polypeptide; or
a2) a nucleic acid comprising a nucleotide sequence encoding a fusion polypeptide comprising: i) a CRISPR-Cas effector polypeptide that exhibits nickase activity; and ii) two or more heterologous polypeptides, wherein one of the two or more heterologous polypeptides is an error-prone DNA polymerase that lacks all or a portion of a 3′-to-5′ exonuclease domain and/or lacks all or a portion of a 5′-to-3′ exonuclease domain, wherein one of the two or more heterologous polypeptides comprises an NES polypeptide, and wherein the two or more heterologous polypeptides does not include an NLS polypeptide; and
b2) one or more guide nucleic acids, wherein one or more guide nucleic acids comprise: i) a targeting region that comprises a nucleotide sequence that binds to a target sequence in the target viral nucleic acid, wherein the nucleotide sequence that binds to the target sequence has a length of from 15 nucleotides to 18 nucleotides; and ii) a protein-binding region that binds to the CRISPR-Cas effector polypeptide; or
a3) a nucleic acid comprising:
1) a first nucleotide sequence encoding a fusion polypeptide comprising: i) a CRISPR-Cas effector polypeptide that exhibits nickase activity; and ii) two or more heterologous polypeptides, wherein one of the two or more heterologous polypeptides is an error-prone DNA polymerase that lacks all or a portion of a 3′-to-5′ exonuclease domain and/or lacks all or a portion of a 5′-to-3′ exonuclease domain, wherein one of the two or more heterologous polypeptides comprises an NES polypeptide, and wherein the two or more heterologous polypeptides does not include an NLS polypeptide; and
2) a second nucleotide sequence encoding one or more guide nucleic acids, wherein one or more guide nucleic acids comprise: i) a targeting region that comprises a nucleotide sequence that binds to a target sequence in the target viral nucleic acid, wherein the nucleotide sequence that binds to the target sequence has a length of from 15 nucleotides to 18 nucleotides; and ii) a protein-binding region that binds to the CRISPR-Cas effector polypeptide.


SEQUENCE LISTING (90 sequences)

SEQ ID NO: 1 - protein, 928 aa, Escherichia coli [sequence omitted]

SEQ ID NO: 2 - protein, 928 aa, synthetic construct
MVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 3 - protein, 928 aa, synthetic construct
MVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 4 - protein, 928 aa, synthetic construct
MVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVFGLPLETVTSEQRRSAKRINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 5 - protein, 928 aa, synthetic construct
MVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVYGLPLETVTSEQRRSAKRINFGLIYGMSAFGLARQLNIPRKEAQKYMDLYFERYHGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 6 - protein, 604 aa, synthetic construct
MSYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVYGLPLETVTSEQRRSAKRINFGLIYGMSAFGLARQLNIPRKEAQKYMDLYFERYHGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 7 - protein, 575 aa, Salasvirus phi29 [sequence omitted]

SEQ ID NO: 8 - protein, 829 aa, Tequintavirus T5
MYSICVTRSCPVVCSKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFDSETSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDIMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKAITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGA

SEQ ID NO: 9 - protein, 683 aa, Vibrio parahaemolyticus
MNERLCYDIETDGLIEEVTKLHCIAIINVDTLEEALYADSLVMMTQGTIKDGLDRLNKASLTLGHNIIKYDHPVINKLTGYELPKDKSFDTLVASRLVFSNIKDIDQGNSGKYTLGKLFGSHSLEAWGIRLGGQQKMEYAPVIDPAQPVYDPTVKPKDAKKDPRWKGSIFTPMMGDYCMQDVRVNVELFRRLERKIHQLGYARSMLLEHEAAWVLAQQERNGFKFDEEKAIKLLGRLAGRREYLYNKLIETFGGWWVSDGVSVPARTINYKDPTKPSRVQGAPFTKVKWVDFNPSSRRHIIKVLTDRGWTPQEFTPSGEAKVDESILKELDFPEARLMAEWFLVQKRLGQLADGNQAWLNTVHPDGFIRGSVNPNGAVTGRATHSFPNVAQVPSCSAEYGAECRELFTVPDGWYLLGSDASGLELRCLANFMARYDDGKYIDVVLNGDIHWSNAQAAGFIPKGTIRDPHNPIHEEARRKAKTFIYAFLYGCGAELTGQQVGWTEEEYLNWKAKGAHKPIINRFKRQGKPWTREKICNILKGEEVQKNFMKGLPALKNLIDECKELHKEQGYIEGIDGRRIYTRSAHASLNTLLQGAGALVCKAWIVEIEKLAIAEGLKHGIDGDFMYCAWVHDEVQIACRTKEIAERIGQLCQVAMTNVEKEFNFVCRLDADFDIGKSWKETH

SEQ ID NO: 10 - protein, 1001 aa, synthetic construct
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVIGSIVSDIEANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGNELRCLAHFMARFDNGEYAHEILNGDIHTKNQIAAELPTRDNAKRFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICH

SEQ ID NO: 11 - protein, 740 aa, Homo sapiens
MEKLGVEPEEEGGGDDDEEDAEAWAMELADVGAAASSQGVHDQVLPTPNASSRVIVHVDLDCFYAQVEMISNPELKDKPLGVQQKYLVVTCNYEARKLGVKKLMNVRDAKEKCPQLVLVNGEDLTRYREMSYKVTELLEEFSPVVERLGFDENFVDLTEMVEKRLQQLQSDELSAVTVSGHVYNNQSINLLDVLHIRLLVGSQIAAEMREAMYNQLGLTGCAGVASNKLLAKLVSGVFKPNQQTVLLPESCQHLIHSLNHIKEIPGIGYKTAKCLEALGINSVRDLQTFSPKILEKELGISVAQRIQKLSFGEDNSPVILSGPPQSFSEEDSFKKCSSEVEAKNKIEELLASLLNRVCQDGRKPHTVRLIIRRYSSEKHYGRESRQCPIPSHVIQKLGTGNYDVMTPMVDILMKLFRNMVNVKMPFHLTLLSVCFCNLKALNTAKKGLIDYYLMPSLSTTSRSGKHSFKMKDTHMEDFPKDKETNRDFLPSGRIESTRTRESPLDTTNFSKEKDINEFPLCSLPEGVDQEVFKQLPVDIQEEILSGKSREKFQGKGSVSCPLHASRGVLSFFSKKQMQDIPINPRDHLSSSKQVSSVSPCEPGTSGFNSSSSSYMSSQKDYSYYLDNRLKDERISQGPKEPQGFHFTNSNPAVSAFHSFPNLQSEQLFSRNHTTDSHKQTVATDSHEGLTENREPDSVDEKITFPSDIDPQVFYELPEAVQKELLAEWKRAGSDFHIGHK

SEQ ID NO: 12 - protein, 713 aa, Homo sapiens
MATGQDRVVALVDMDCFFVQVEQRQNPHLRNKPCAVVQYKSWKGGGIIAVSYEARAFGVTRSMWADDAKKLCPDLLLAQVRESRGKANLTKYREASVEVMEIMSRFAVIERASIDEAYVDLTSAVQERLQKLQGQPISADLLPSTYIEGLPQGPTTAEETVQKEGMRKQGLFQWLDSLQIDNLTSPDLQLTVGAVIVEEMRAAIERETGFQCSAGISHNKVLAKLACGLNKPNRQTLVSHGSVPQLFSQMPIRKIRSLGGKLGASVIEILGIEYMGELTQFTESQLQSHFGEKNGSWLYAMCRGIEHDPVKPRQLPKTIGCSKNFPGKTALATREQVQWWLLQLAQELEERLTKDRNDNDRVATQLVVSIRVQGDKRLSSLRRCCALTRYDAHKMSHDAFTVIKNCNTSGIQTEWSPPLTMLFLCATKFSASAPSSSTDITSFLSSDPSSLPKVPVTSSEAKTQGSGPAVTATKKATTSLESFFQKAAERQKVKEASLSSLTAPTQAPMSNSPSKPSLPFQTSQSTGTEPFFKQKSLLLKQKQLNNSSVSSPQQNPWSNCKALPNSLPTEYPGCVPVCEGVSKLEESSKATPAEMDLAHNSQSMHASSASKSVLEVTQKATPNPSLLAAEDQVPCEKCGSLVPVWDMPEHMDYHFALELQKSFLQPHSSNPQVVSAVSHQGKRNPKSPLACTNKRPRPEGMQTLESFFKPLTH

SEQ ID NO: 13 - protein, 870 aa, Homo sapiens
MDSTKEKCDSYKDDLLLRMGLNDNKAGMEGLDKEKINKIIMEATKGSRFYGNELKKEKQVNQRIENMMQQKAQITSQQLRKAQLQVDRFAMELEQSRNLSNTIVHIDMDAFYAAVEMRDNPELKDKPIAVGSMSMLSTSNYHARRFGVRAAMPGFIAKRLCPQLIIVPPNFDKYRAVSKEVKEILADYDPNFMAMSLDEAYLNITKHLEERQNWPEDKRRYFIKMGSSVENDNPGKEVNKLSEHERSISPLLFEESPSDVQPPGDPFQVNFEEQNNPQILQNSVVFGTSAQEVVKEIRFRIEQKTTLTASAGIAPNTMLAKVCSDKNKPNGQYQILPNRQAVMDFIKDLPIRKVSGIGKVTEKMLKALGIITCTELYQQRALLSLLFSETSWHYFLHISLGLGSTHLTRDGERKSMSVERTFSEINKAEEQYSLCQELCSELAQDLQKERLKGRTVTIKLKNVNFEVKTRASTVSSVVSTAEEIFAIAKELLKTEIDADFPHPLRLRLMGVRISSFPNEEDRKHQQRSIIGFLQAGNQALSATECTLEKTDKDKFVKPLEMSHKKSFFDKKRSERKWSHQDTFKCEAVNKQSFQTSQPFQVLKKKMNENLEISENSDDCQILTCPVCFRAQGCISLEALNKHVDECLDGPSISENFKMFSCSHVSATKVNKKENVPASSLCEKQDYEAHPKIKEISSVDCIALVDTIDNSSKAESIDALSNKHSKEECSSLPSKSFNIEHCHQNSSSTVSLENEDVGSFRQEYRQPYLCEVKTGQALVCPVCNVEQKTSDLTLFNVHVDVCLNKSFIQELRKDKFNPVNQPKESSRSTGSSSGVQKAVTRTKRPGLMTKYSTSKKIKPNNPKHTLDIFFK

SEQ ID NO: 14 - protein, 2590 aa, Homo sapiens
MNLLRRSGKRRRSESGSDSFSGSGGDSSASPQFLSGSVLSPPPGLGRCLKAAAAGECKPTVPDYERDKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEMGVQWNPCALLHSSTCSLTHSESEVKEHTFISQTKSSYKKLTSKNKSNTIFSDSYIKHSPNIVQDLNKSREHTSSFNCNFQNGNQEHQTCSIFRARKRASLDINKEKPGASQNEGKTSDKKVVQTFSQKTKKAPLNFNSEKMSRSFRSWKRRKHLKRSRDSSPLKDSGACRIHLQGQTLSNPSLCEDPFTLDEKKTEFRNSGPFAKNVSLSGKEKDNKTSFPLQIKQNCSWNITLTNDNFVEHIVTGSQSKNVTCQATSVVSEKGRGVAVEAEKINEVLIQNGSKNQNVYMKHHDIHPINQYLRKQSHEQTSTITKQKNIIERQMPCEAVSSYINRDSNVTINCERIKLNTEENKPSHFQALGDDISRTVIPSEVLPSAGAFSKSEGQHENFLNISRLQEKTGTYTTNKTKNNHVSDLGLVLCDFEDSFYLDTQSEKIIQQMATENAKLGAKDTNLAAGIMQKSLVQQNSMNSFQKECHIPFPAEQHPLGATKIDHLDLKTVGTMKQSSDSHGVDILTPESPIFHSPILLEENGLFLKKNEVSVTDSQLNSFLQGYQTQETVKPVILLIPQKRTPTGVEGECLPVPETSLNMSDSLLFDSFSDDYLVKEQLPDMQMKEPLPSEVTSNHFSDSLCLQEDLIKKSNVNENQDTHQQLTCSNDESIIFSEMDSVQMVEALDNVDIFPVQEKNHTVVSPRALELSDPVLDEHHQGDQDGGDQDERAEKSKLTGTRQNHSFIWSGASFDLSPGLQRILDKVSSPLENEKLKSMTINFSSLNRKNTELNEEQEVISNLETKQVQGISFSSNNEVKSKIEMLENNANHDETSSLLPRKESNIVDDNGLIPPTPIPTSASKLTFPGILETPVNPWKTNNVLQPGESYLFGSPSDIKNHDLSPGSRNGFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 15 - protein, 900 aa, Homo sapiens
MENYEALVGFDLCNTPLSSVAQKIMSAMHSGDLVDSKTWGKSTETMEVINKSSVKYSVQLEDRKTQSPEKKDLKSLRSQTSRGSAKLSPQSFSVRLTDQLSADQKQKSISSLTLSSCLIPQYNQEASVLQKKGHKRKHFLMENINNENKGSINLKRKHITYNNLSEKTSKQMALEEDTDDAEGYLNSGNSGALKKHFCDIRHLDDWAKSQLIEMLKQAAALVITVMYTDGSTQLGADQTPVSSVRGIVVLVKRQAEGGHGCPDAPACGPVLEGFVSDDPCIYIQIEHSAIWDQEQEAHQQFARNVLFQTMKCKCPVICFNAKDFVRIVLQFFGNDGSWKHVADFIGLDPRIAAWLIDPSDATPSFEDLVEKYCEKSITVKVNSTYGNSSRNIVNQNVRENLKTLYRLTMDLCSKLKDYGLWQLFRTLELPLIPILAVMESHAIQVNKEEMEKTSALLGARLKELEQEAHFVAGERFLITSNNQLREILFGKLKLHLLSQRNSLPRTGLQKYPSTSEAVLNALRDLHPLPKIILEYRQVHKIKSTFVDGLLACMKKGSISSTWNQTGTVTGRLSAKHPNIQGISKHPIQITTPKNFKGKEDKILTISPRAMFVSSKGHTFLAADFSQIELRILTHLSGDPELLKLFQESERDDVFSTLTSQWKDVPVEQVTHADREQTKKVVYAVVYGAGKERLAACLGVPIQEAAQFLESFLQKYKKIKDFARAAIAQCHQTGCVVSIMGRRRPLPRIHAHDQQLRAQAERQAVNFVVQGSAADLCKLAMIHVFTAVAASHTLTARLVAQIHDELLFEVEDPQIPECAALVRRTMESLEQVQALELQLQVPLKVSLSAGRSWGHLVPLQEAWGPPPGPCRTESPSNSLAAPGSPASTQPPPLHFSPSFCL

SEQ ID NO: 16 - protein, 1368 aa, Streptococcus pyogenes [sequence omitted]

SEQ ID NO: 17 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 18 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 19 - protein, 1306 aa, Acidaminococcus sp.
  source 1..1306: note = Acidaminococcus sp. BV3L6
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 20 - protein, 1228 aa, unidentified
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 21 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 22 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 23 - protein, 1601 aa, Desulfonema ishimotonii
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 24 - protein, 966 aa, synthetic construct
IEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 25 - protein, 1082 aa, Prevotella sp.
  source 1..1082: note = provisionally referred to as Prevotella pectinovora
NQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMK

SEQ ID NO: 26 - protein, 1320 aa, Prevotella sp.
  source 1..1320: note = provisionally referred to as Prevotella ihumii
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSKFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKFKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSNSGTQFGGYLFRKKNEIGEYDYFLGISSKTQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIGNLLKTISCLKNKEEFHDLIKKDYQIFTEVQAVIDEICKQKTFIYFPVSNVELEKEMGDKDKPLCLFQISNKDLSFAKTFSANLRKKRGAENLHTMLFKALMSGNQDNLDLGSGAIFYRAKSLDGNKPTHPANEAIKCRNVANKDKVSLFTYDIYKNRRYMENKFLFHLSIVQNYKAAKDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGEMFVTRGQKIEKAVYQQFEKSLVDKLSYLVDKKRPYNELGGILKAYQLASSITKSNSDKQNGFLFYVPAWNTSKIDPVTGFTDLLRPKAMTIKEAQDFFGAFDNISYNDKGYFEFETNYDKFKIRMKGAQTRWTICTFGNRIKHKKDKNYWNYEEVELTEEFKKLFDKYDIDYRDGNLKEEILKIDNRKFFDALIKLLQLTLQLRNSDDKGNDYIVSPIANADGKFFNSNDGCKELPLDADANGAYNIARKGLWVVRQIKDKKDKISKLSNQEWLKFAQEKPYLK

SEQ ID NO: 27 - protein, 255 aa, Prevotella disiens
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDV

SEQ ID NO: 28 - protein, 15 aa, synthetic construct
TLVTTLTHGVQCFSR

SEQ ID NO: 29 - DNA, 45 bp, synthetic construct
  source 1..45: mol_type = other DNA
accctcgtgaccaccctgacccatggcgtgcagtgcttcagccgc

SEQ ID NO: 30 - protein, 15 aa, synthetic construct
TLVTTLTYGVQCFSR

SEQ ID NO: 31 - DNA, 45 bp, synthetic construct
  source 1..45: mol_type = other DNA
accctcgtgaccaccctgacctatggcgtgcagtgcttcagccgc

SEQ ID NO: 32 - protein, 1250 aa, synthetic construct
MDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 33 - DNA, 1812 bp, synthetic construct
  source 1..1812: mol_type = other DNA
tcttacgataattacgttaccatcctcgacgaagagaccttgaaagcttggatagcgaaactcgaaaaggccccggtcttcgcctttgacacggagaccgacagtttggacaacatcagtgccaacctcgttggtctgtcctttgccattgaacctggggtagctgcatacatacccgttgctcatgactatctcgatgccccggatcagatctcccgagagcgggcgctcgagctgttgaagcctctgctcgaagacgaaaaggcgctgaaagtcggtcagaacctgaagtatgccaggggcattctggcgaactatggaattgaactcagggggattgcatttgacacgatgctcgaaagctatatcttgaattcagtcgctgggaggcatgatatggattctttggccgagagatggctgaaacataagacgatcacgttcgaagagatcgccggaaagggcaagaaccagctcacattcaatcagattgctttggaggaagccggtagatatgcggctgaagacgctgatgtaacacttcagttgcaccttaaaatgtggccagacctccaaaaacataaaggcccgctcaacgtgtttgagaacatcgagatgcccctggtccccgtattgtcaagaattgaacggaatggagttaagattgacccgaaagtcctgcacaaccattccgaggaattgactcttcgactcgctgaattggagaagaaagcccatgagattgccggtgaagagtttaacctttctagtacaaaacaactccaaactatattgtttgagaaacaggggatcaaacctcttaaaaaaacgccagggggtgccccaagcacaagcgaagaggtgcttgaggaattggcgctcgactatccattgccgaaggtcatcctggagtatcgcggtcttgctaagttgaagtctacttatacggataagttgccacttatgatcaacccgaaaaccggtcgcgtccacacctcatatcaccaggctgttactgctacgggacgattgtcttccacggacccaaaccttcagaacatccctgttagaaatgaagaggggagaaggatcagacaggcgttcatcgcacctgaggattacgttattgtgtctgcggattattcacagaacgagttgaggattatggcgcacttgagcagggataaaggccttttgacagcatttgccgagggcaaagatatacaccgagcaactgcggcggaagtatatggtcttcctctggagaccgtcactagtgaacagcggcgatccgctaaacggataaattttggcctcatttacgggatgagcgccttcgggctggcacgacagctgaatatccccaggaaagaagcacagaaatacatggatttgtactttgagcgatatcacggggttctggagtatatggaaagaaccagggcgcaagcaaaagagcaggggtacgttgaaaccttggacggtcgacggttgtatcttccagacatcaaatcatccaacggtgcgcgccgggcggcagctgaacgagctgctataaacgcccctatgcagggcaccgccgcagacattatcaagagggccatgatcgcagtggatgcttggttgcaagcagagcaacctagggtccgaatgatcatgcaggtacacgacgaattggttttcgaagtccacaaagacgatgtcgatgcggtcgccaagcagattcatcaactcatggagaattgtacacggcttgacgtccccctcctggtagaagtcggaagcggtgagaactgggaccaggcccactaa

SEQ ID NO: 34 - protein, 9 aa, synthetic construct
LPPLERLTL

SEQ ID NO: 35 - protein, 10 aa, synthetic construct
LALKLAGLDL

SEQ ID NO: 36 - protein, 14 aa, synthetic construct
MEELSQALASSFSV

SEQ ID NO: 37 - protein, 14 aa, synthetic construct
EAETVSAMALLSVG

SEQ ID NO: 38 - protein, 14 aa, synthetic construct
ELDELMASLSDFKF

SEQ ID NO: 39 - protein, 11 aa, synthetic construct
VDQLRLERLQI

SEQ ID NO: 40 - protein, 9 aa, synthetic construct
IDLSGLTLQ

SEQ ID NO: 41 - protein, 10 aa, synthetic construct
LRALERLQID

SEQ ID NO: 42 - protein, 10 aa, synthetic construct
LQKKLEELEL

SEQ ID NO: 43 - protein, 10 aa, synthetic construct
MQELSNILNL

SEQ ID NO: 44 - protein, 10 aa, synthetic construct
LCQAFSDVIL

SEQ ID NO: 45 - protein, 17 aa, synthetic construct
RTFDMHSLESSLIDIMR

SEQ ID NO: 46 - protein, 17 aa, synthetic construct
TNLEALQKKLEELELDE

SEQ ID NO: 47 - protein, 17 aa, synthetic construct
RSFEMTEFNQALEEIKG

SEQ ID NO: 48 - protein, 12 aa, synthetic construct
PLQLPPLERLTL

SEQ ID NO: 49 - protein, 12 aa, synthetic construct
NELALKLAGLDI

SEQ ID NO: 50 - protein, 15 aa, synthetic construct
ERFEMFRELNEALEL

SEQ ID NO: 51 - protein, 15 aa, synthetic construct
DHAEKVAEKLEALSV

SEQ ID NO: 52 - protein, 15 aa, synthetic construct
QLVEELLKIICAFQL

SEQ ID NO: 53 - protein, 15 aa, synthetic construct
TNLEALQKKLEELEL

SEQ ID NO: 54 - protein, 15 aa, synthetic construct
DVKEEMTSALATMRV

SEQ ID NO: 55 - protein, 15 aa, synthetic construct
STNGSLAAEFRHLQL

SEQ ID NO: 56 - protein, 15 aa, synthetic construct
PSVQELTEQIHRLLM

SEQ ID NO: 57 - protein, 15 aa, synthetic construct
MNFKELKDFLKELNI

SEQ ID NO: 58 - protein, 15 aa, synthetic construct
ENFEILMKLKESLEL

SEQ ID NO: 59 - protein, 15 aa, synthetic construct
FETVYELTKMCTIRM

SEQ ID NO: 60 - protein, 15 aa, synthetic construct
SGKASSSLGLQDFDL

SEQ ID NO: 61 - protein, 15 aa, synthetic construct
PKYSDIDVDGLCSEL

SEQ ID NO: 62 - protein, 15 aa, synthetic construct
VDLACTPTDVRDVDI

SEQ ID NO: 63 - protein, 11 aa, synthetic construct
YGRKKRRQRRR

SEQ ID NO: 64 - protein, 12 aa, synthetic construct
RRQRRTSKLMKR

SEQ ID NO: 65 - protein, 27 aa, synthetic construct
GWTLNSAGYLLGKINLKALAALAKKIL

SEQ ID NO: 66 - protein, 33 aa, synthetic construct
KALAWEAKLAKALAKALAKHLAKALAKALKCEA

SEQ ID NO: 67 - protein, 16 aa, synthetic construct
RQIKIWFQNRRMKWKK

SEQ ID NO: 68 - protein, 9 aa, synthetic construct
RKKRRQRRR

SEQ ID NO: 69 - protein, 8 aa, synthetic construct
RKKRRQRR

SEQ ID NO: 70 - protein, 11 aa, synthetic construct
YARAAARQARA

SEQ ID NO: 71 - protein, 11 aa, synthetic construct
THRLPRRRRRR

SEQ ID NO: 72 - protein, 11 aa, synthetic construct
GGRRARRRRRR

SEQ ID NO: 73 - protein, 335 aa, Homo sapiens
MSKRKAPQETLNGGITDMLTELANFEKNVSQAIHKYNAYRKAASVIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSE

SEQ ID NO: 74 - protein, 445 aa, Homo sapiens
MEKLGVEPEEEGGGDDDEEDAEAWAMELADVGAAASSQGVHDQVLPTPNASSRVIVHVDLDCFYAQVEMISNPELKDKPLGVQQKYLVVTCNYEARKLGVKKLMNVRDAKEKCPQLVLVNGEDLTRYREMSYKVTELLEEFSPVVERLGFDENFVDLTEMVEKRLQQLQSDELSAVTVSGHVYNNQSINLLDVLHIRLLVGSQIAAEMREAMYNQLGLTGCAGVASNKLLAKLVSGVFKPNQQTVLLPESCQHLIHSLNHIKEIPGIGYKTAKCLEALGINSVRDLQTFSPKILEKELGISVAQRIQKLSFGEDNSPVILSGPPQSFSEEDSFKKCSSEVEAKNKIEELLASLLNRVCQDGRKPHTVRLIIRRYSSEKHYGRESRQCPIPSHVIQKLGTGNYDVMTPMVDILMKLFRNMVNVKMPFHLTLLSVCFCNLKALNTAK

SEQ ID NO: 75 - protein, 419 aa, Homo sapiens
ELADVGAAASSQGVHDQVLPTPNASSRVIVHVDLDCFYAQVEMISNPELKDKPLGVQQKYLVVTCNYEARKLGVKKLMNVRDAKEKCPQLVLVNGEDLTRYREMSYKVTELLEEFSPVVERLGFDENFVDLTEMVEKRLQQLQSDELSAVTVSGHVYNNQSINLLDVLHIRLLVGSQIAAEMREAMYNQLGLTGCAGVASNKLLAKLVSGVFKPNQQTVLLPESCQHLIHSLNHIKEIPGIGYKTAKCLEALGINSVRDLQTFSPKILEKELGISVAQRIQKLSFGEDNSPVILSGPPQSFSEEDSFKKCSSEVEAKNKIEELLASLLNRVCQDGRKPHTVRLIIRRYSSEKHYGRESRQCPIPSHVIQKLGTGNYDVMTPMVDILMKLFRNMVNVKMPFHLTLLSVCFCNLKALNTAK

SEQ ID NO: 76 - protein, 899 aa, Homo sapiens
ENYEALVGFDLCNTPLSSVAQKIMSAMHSGDLVDSKTWGKSTETMEVINKSSVKYSVQLEDRKTQSPEKKDLKSLRSQTSRGSAKLSPQSFSVRLTDQLSADQKQKSISSLTLSSCLIPQYNQEASVLQKKGHKRKHFLMENINNENKGSINLKRKHITYNNLSEKTSKQMALEEDTDDAEGYLNSGNSGALKKHFCDIRHLDDWAKSQLIEMLKQAAALVITVMYTDGSTQLGADQTPVSSVRGIVVLVKRQAEGGHGCPDAPACGPVLEGFVSDDPCIYIQIEHSAIWDQEQEAHQQFARNVLFQTMKCKCPVICFNAKDFVRIVLQFFGNDGSWKHVADFIGLDPRIAAWLIDPSDATPSFEDLVEKYCEKSITVKVNSTYGNSSRNIVNQNVRENLKTLYRLTMDLCSKLKDYGLWQLFRTLELPLIPILAVMESHAIQVNKEEMEKTSALLGARLKELEQEAHFVAGERFLITSNNQLREILFGKLKLHLLSQRNSLPRTGLQKYPSTSEAVLNALRDLHPLPKIILEYRQVHKIKSTFVDGLLACMKKGSISSTWNQTGTVTGRLSAKHPNIQGISKHPIQITTPKNFKGKEDKILTISPRAMFVSSKGHTFLAADFSQIELRILTHLSGDPELLKLFQESERDDVFSTLTSQWKDVPVEQVTHADREQTKKVVYAVVYGAGKERLAACLGVPIQEAAQFLESFLQKYKKIKDFARAAIAQCHQTGCVVSIMGRRRPLPRIHAHDQQLRAQAERQAVNFVVQGSAADLCKLAMIHVFTAVAASHTLTARLVAQIHDELLFEVEDPQIPECAALVRRTMESLEQVQALELQLQVPLKVSLSAGRSWGHLVPLQEAWGPPPGPCRTESPSNSLAAPGSPASTQPPPLHFSPSFCL

SEQ ID NO: 77 - protein, 511 aa, Homo sapiens
MATGQDRVVALVDMDCFFVQVEQRQNPHLRNKPCAVVQYKSWKGGGIIAVSYEARAFGVTRSMWADDAKKLCPDLLLAQVRESRGKANLTKYREASVEVMEIMSRFAVIERASIDEAYVDLTSAVQERLQKLQGQPISADLLPSTYIEGLPQGPTTAEETVQKEGMRKQGLFQWLDSLQIDNLTSPDLQLTVGAVIVEEMRAAIERETGFQCSAGISHNKVLAKLACGLNKPNRQTLVSHGSVPQLFSQMPIRKIRSLGGKLGASVIEILGIEYMGELTQFTESQLQSHFGEKNGSWLYAMCRGIEHDPVKPRQLPKTIGCSKNFPGKTALATREQVQWWLLQLAQELEERLTKDRNDNDRVATQLVVSIRVQGDKRLSSLRRCCALTRYDAHKMSHDAFTVIKNCNTSGIQTEWSPPLTMLFLCATKFSASAPSSSTDITSFLSSDPSSLPKVPVTSSEAKTQGSGPAVTATKKATTSLESFFQKAAERQKVKEASLSSLTAPTQAPMSN

SEQ ID NO: 78 - protein, 560 aa, synthetic construct
MDSTKEKCDSYKDDLLLRMGLNDNKAGMEGLDKEKINKIIMEATKGSRFYGNELKKEKQVNQRIENMMQQKAQITSQQLRKAQLQVDRFAMELEQSRNLSNTIVHIDMDAFYAAVEMRDNPELKDKPIAVGSMSMLSTSNYHARRFGVRAAMPGFIAKRLCPQLIIVPPNFDKYRAVSKEVKEILADYDPNFMAMSLDEAYLNITKHLEERQNWPEDKRRYFIKMGSSVENDNPGKEVNKLSEHERSISPLLFEESPSDVQPPGDPFQVNFEEQNNPQILQNSVVFGTSAQEVVKEIRFRIEQKTTLTASAGIAPNTMLAKVCSDKNKPNGQYQILPNRQAVMDFIKDLPIRKVSGIGKVTEKMLKALGIITCTELYQQRALLSLLFSETSWHYFLHISLGLGSTHLTRDGERKSMSVERTFSEINKAEEQYSLCQELCSELAQDLQKERLKGRTVTIKLKNVNFEVKTRASTVSSVVSTAEEIFAIAKELLKTEIDADFPHPLRLRLMGVRISSFPNEEDRKHQQRSIIGFLQAGNQALSATECTLEKTDKDKFVKPLE

SEQ ID NO: 79 - protein, 5 aa, synthetic construct
  VARIANT >5: note = The entire sequence of amino acids 1-5 can be repeated one or more times
GSGGS

SEQ ID NO: 80 - protein, 6 aa, synthetic construct
  VARIANT >6: note = The entire sequence of amino acids 1-6 can be repeated one or more times
GGSGGS

SEQ ID NO: 81 - protein, 5 aa, synthetic construct
  VARIANT >5: note = The entire sequence of amino acids 1-5 can be repeated one or more times
GGGGS

SEQ ID NO: 82 - protein, 4 aa, synthetic construct
GGSG

SEQ ID NO: 83 - protein, 5 aa, synthetic construct
GGSGG

SEQ ID NO: 84 - protein, 5 aa, synthetic construct
GSGSG

SEQ ID NO: 85 - protein, 5 aa, synthetic construct
GSGGG

SEQ ID NO: 86 - protein, 5 aa, synthetic construct
GGGSG

SEQ ID NO: 87 - protein, 5 aa, synthetic construct
GSSSG

SEQ ID NO: 88 - protein, 1368 aa, synthetic construct
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 89 - protein, 1368 aa, synthetic construct
MDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 90 - protein, 1250 aa, synthetic construct
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD